SEQUENCE LISTING (4 sequences)

SEQ ID NO: 1 - DNA, 26 bp, Artificial
  Synthetic oligonucleotide primer
gccgccatggatggcaaccgcagcag

SEQ ID NO: 2 - DNA, 20 bp, Artificial
  Synthetic oligonucleotide primer
ggtctttaccctacctgact

SEQ ID NO: 3 - DNA, 24 bp, Artificial
  Synthetic oligonucleotide primer
ctggccatagaaccagagaagtga

SEQ ID NO: 4 - DNA, 24 bp, Artificial
  Synthetic oligonucleotide primer
ccacctctgccaacgggtcccgcg

(19) United States
(12) Patent Application Publication
(10) Pub. No.: US 2008/0108582 A1
(43) Pub. Date: May 8, 2008

(54) Methods And Compositions For Combinatorial Approaches To Cancer Gene Therapy

(76) Inventor: Irene Kazhdan, San Antonio, TX (US)

Correspondence Address: MYERS BIGEL SIBLEY & SAJOVEC, PO BOX 37428, RALEIGH, NC, 27627, US

(21) Appl. No.: 10/578,445

(22) PCT Filed: Oct. 27, 2004
(86) PCT No.: PCT/US04/35643

§ 371 Date: May 14, 2007

Related U.S. Application Data
(60) Provisional application No. 60/519,342, filed on Nov. 12, 2003.

(51) Int. Cl. A61K 31/7088; C07H 21/00; C12N 15/63; A61P 35/00; C12N 15/64

(52) U.S. Cl. 514/44; 536/23.1; 435/320.1

(57) ABSTRACT

The present invention provides a nucleic acid comprising a) a nucleotide sequence encoding one or more pro-apoptotic proteins, and b) a nucleotide sequence encoding one or more tumor-specific and/or tissue-specific promoters. Also provided is a method of treating cancer, comprising administering the compositions of this invention to a subject.

### STATEMENT OF PRIORITY

This application claims the benefit, under 35 U.S.C. § 119(e), of U.S. Provisional Application Ser. No. 60/519,342, filed Nov. 12, 2003, the entire contents of which are incorporated by reference herein.

### STATEMENT OF GOVERNMENT SUPPORT

Research directed to this invention is supported in part by National Institutes of Health (NIH) Grant No. NIH 5 K12 CA01723-10 and by American Cancer Society Grant No. IRG-00-173-01. The Government has certain rights in this invention.

### FIELD OF THE INVENTION

The present invention is directed to compositions and methods of their use in the induction of apoptosis in tumor cells as a treatment of cancer.

### BACKGROUND OF THE INVENTION

In recent years, tremendous progress has been made in identifying genetic alterations in cancer cells. This knowledge has been used extensively to devise therapeutic interventions, which preferentially affect tumors and spare normal tissues. Despite these efforts, effective molecular therapies for cancer are still scarce; therefore, development and pre-clinical assessment of new treatment modalities is of paramount importance. One of such approaches is gene therapy.

Success of gene therapy depends on the ability to select efficient “killer genes” and to target their expression selectively to cancer cells. Among promising killer genes are those that induce apoptosis, which is also known as programmed cell death.

Apoptosis is a process employed by multicellular organisms to eliminate redundant, damaged or potentially harmful cells. Apoptotic signaling can initiate from within the cell (intrinsic pathway) or from outside (extrinsic pathway).

In the intrinsic or mitochondrial pathway, intracellular stimuli signaling cell damage are delivered to the mitochondria. Whether the signal is transmitted further depends on the balance of pro- and anti-survival forces. Pro-survival members of the bcl-2 family (bcl-2, bcl-XL, bclw, etc.) protect mitochondrial integrity and interfere with transmission of the apoptotic stimulus. Pro-apoptotic members of the same family (Bax, Bak, bcl-Xs, Bik, Bad, etc.) counteract activity of pro-survival genes and mediate loss of mitochondrial potential and release of cytochrome c, ATP and LAP inhibitor, Smac, into the cytoplasm. There, cytochrome c activates Apafl, which recruits and activates caspase 9, which subsequently activates caspase 3. Activated caspases can be blocked by IAP (inhibitors of apoptosis) family members XIAP, c-IAP1 and 2, and survivin. The IAP block is counteracted by Smac.

The extrinsic or death receptor (DR) pathway is initiated by binding of ligand to death receptor, leading to receptor oligomerization. Several members of the DR family have been characterized. They include Fas, which interacts with Fas ligand, tumor necrosis factor (TNF) receptor, and Death Receptors 4 and 5, which interact with TNF-related apoptosis inducing ligand (TRAIL). Receptor oligomerization results in assembly of the DISC (death-inducing signaling complex), which includes, among others, the adaptor molecule, FADD and caspase 8. Caspase 8, activated inside the DISC, subsequently activates effector caspases and also cleaves the bcl-2 family member, Bic, which then carries the apoptotic signal to mitochondria.

The relative contribution of the direct effector caspase activation and the mitochondrial pathway to transmission of apoptotic stimulus from DRs is cell-type dependent. Type I cells, which are mostly of lymphoid origin, preferentially utilize direct activation of effector caspases. Breast cancer cell line McF-7 is representative of Type II cells, which depend on mitochondria for induction of programmed cell death.

There are many genes that function as pro-apoptotic genes or “killer genes” and destroy cancer cells. One such gene, tBid, is a part of a large group of genes that are expressed to kill damaged cells in the body, via apoptosis, before they become harmful. Because tBid is toxic to normal cells as well, its effects are ideally limited to cancer cells. The approach used in gene therapy to achieve this type of selective action is called transcriptional targeting.

Transcriptional targeting uses DNA elements, called promoters, which regulate gene transcription. Some of these promoters are very active in tumor cells, but not in normal ones. These promoters can be used to drive production of “killer gene” products instead, and thus destroy cancer cells specifically. One of the problems with this approach is the fact that different tumors, and different cancer cells within a tumor, vary in the patterns of gene activity. Thus a single promoter can be active in one cell of a tumor and not active in another cell of the same tumor. This means that if a single promoter is used to drive expression of a killer gene, not all the tumor cells will produce the killer gene. Thus, a combinatorial approach is needed, wherein several different promoters, which are active in a large proportion of tumor cells overall, are used in combination to direct the killer gene to destroy most, if not all, cells in the same tumor. Three such promoters that match these criteria are Survivin, hTERT and Muc1.

Survivin, a member of the LAP anti-apoptotic gene family, is highly and consistently overexpressed in neoplastic cells of diverse origin. Its expression correlated with poor prognosis in a majority of cells of cancer types investigated. Survivin is undetectable in normal adult tissues except small proportions of CD34+ hematopoietic cells, T-lymphocytes and colonic mucosa. In Survivin-positive tumors, a fraction of cells expressing the protein varies between 50 and 100%. In breast neoplasms, Survivin is detected in 70% of samples. Its expression is at least partially regulated at the transcriptional level. In vivo analysis of the Survivin promoter demonstrated that it was preferentially active in tumor cells. The hTERT gene encodes a catalytic subunit of human telomerase. Telomerase activity is detected in a high proportion of human tumors and is absent from a majority of normal adult tissues with the exception of a fraction of actively proliferating hematopoietic, epithelial and germ cells. Evaluation of malignant breast neoplasms demonstrated varying levels of telomerase enzymatic activity in 72 to 96% of samples. The expression of hTERT is controlled at least in part at the transcriptional level. The hTERT promoter was shown to be effective in driving tumor-specific gene therapy in telomerase positive neoplastic cell lines.

DF3/Muc-1 is a gene normally expressed in small subpopulations of epithelial cells from various tissues. It is overexpressed in up to 70% of breast tumors and in DF3/Muc1 positive tumors, the fraction of cells expressing the protein varies between patients, with a median of 30%. The Muc1 promoter was shown to target β-galactosidase expression to Muc1-positive breast cancer cells both in vitro and in vivo.

The present invention overcomes previous problems in the art by providing methods and compositions wherein a combination of tumor- and/or tissue-specific promoters direct the expression of various killer genes to destroy tumor cells and treat cancers.

### SUMMARY OF THE INVENTION

The present invention provides a nucleic acid comprising: a) a first nucleotide sequence encoding one or more pro-apoptotic proteins, and b) a second nucleotide sequence encoding a tumor- and/or tissue-specific promoter functionally (operably) linked to and directing expression of the first nucleotide sequence.

The present invention further provides a method of treating a cancer in a subject, comprising administering to the subject an effective amount of one or more compositions of this invention either simultaneously or sequentially in any order.

Furthermore, the present invention provides a method of treating a cancer in a subject, comprising: a) identifying overexpressed tumor- and/or tissue-specific proteins in tumor cells of the subject and/or promoters with high activity in tumor cells of the subject; b) producing a nucleic acid comprising a first nucleotide sequence comprising one or more of the promoters of the overexpressed tumor- and/or tissue-specific proteins identified in step (a) and/or comprising a first nucleotide sequence comprising one or more of the promoters identified in step (a) as having high activity, and a second nucleotide sequence encoding one or more pro-apoptotic proteins functionally (operably) linked to the first nucleotide sequence; and c) administering an effective amount of the nucleic acid of step (b) to the subject, thereby treating cancer in the subject.

Additionally provided is a method of producing a viral vector comprising a nucleic acid of this invention, comprising introducing into a packaging cell an siRNA construct and/or an antisense sequence that targets a nucleotide sequence encoding a pro-apoptotic protein, either prior to, or simultaneously with introducing into the packaging cell the corresponding nucleotide sequence encoding one or more pro-apoptotic proteins, operably linked to one or more tumor- and/or tissue-specific promoters.

### BRIEF DESCRIPTION OF THE DRAWINGS

FIGS. 1A-C. Killing efficiency of CMV-DR4. A. Cells were plated in duplicates and co-transfected at a ratio of 1:1 with a β-galactosidase reporter plasmid and pcDNA3.1 vector (control) or pcD-DR4. β-galactosidase activity was determined 36 hours later. Cell survival was measured as a percent of β-galactosidase activity in pcD-DR4-transfected cultures relative to vector-transfected control cultures. The data represent an average of at least three independent experiments. B. Cells were transfected as described above and either left untreated, or treated with 100 ng of TRAIL at the time of transfection. β-galactosidase activity was measured 36 hours post transfection. Relative cell survival was determined as described above. C. CAMA-1 cells were transfected with IRES2-EGFP constructs. Unmodified vector and IRES-Luc, in which luciferase gene served as a surrogate “killer gene” were used as controls to IRES-DR4. Thirty-six hours after transfection, the proportions of GFP positive cells were determined by flow cytometry.

FIGS. 2A-B. Effect of TRAIL on breast cancer cell lines. A. Breast cancer cells were plated in triplicate wells and treated with increasing concentrations of TRAIL and PBS-treated cells served as control. Cytotoxicity was determined by MTS test 36 hours later. The data represent an average of three independent experiments. B. Cells were left untreated or treated with TRAIL at a concentration of 100 ng/ml. Thirty-six hours later cells were harvested, stained with PE-labeled Annexin V and analyzed by flow cytometry.

FIGS. 3A-B. Activity of hTERT promoter in breast cancer cell lines. A. Evaluation of hTERT promoter activity. Cells in duplicate wells were co-transfected with phTERT and pRL-TK plasmids at a ratio of 1:1. A Dual luciferase assay was performed 36 hours later. The data are presented as a ratio of hTERT-guided firefly luciferase activity to RL activity and represent an average of at least three independent experiments. B. hTERT promoter activity is significantly lower than that of CMV promoter in breast cancer cells. Cells were transfected with pRL-TK together with either phTERT or pcD-Luc construct (in which firefly luciferase is guided by a CMV promoter) as described above. The data are presented as a ratio of hTERT-guided firefly luciferase activity to RL activity.

FIGS. 4A-B. Killing efficiency of hTERT-DR4 in breast cancer cell lines with relatively high hTERT promoter activity (A) and absence of toxicity in cells with low hTERT promoter activity (B). Cells were transfected with β-galactosidase together with hTERT-DR4 or hTERT-Luciferase and incubated for 36 hours with or without 100 ng of TRAIL. Cell survival was measured as a percent of β-galactosidase activity in hTERT-DR4-transfected cultures relative to vector-transfected cultures.

FIG. 5. Expression of DR4, DR5, TRAIL and FLIP isoforms in breast cancer cell lines. Total protein extracts were prepared from exponentially growing cells. 25 μg of whole cell protein lysate from each sample was subjected to Western blot analysis as described herein. Equal loading is demonstrated by actin protein levels.

Lanes: 1. MCF-7 cells; 2. T-47D cells; 3. HCC 1937 cells; 4. MDA-MB-231 cells; 5.

CAMA-1 cells; 6. AU 562 cells.

FIGS. 6A-B. Expression of caspases 3 and 7, bcl-XL and bcl-2 in breast cancer cell lines. A. Messenger RNA levels for caspases 3 and 7, bcl-XL and bcl-2 were assessed by RNase protection assay. Total RNA was extracted from exponentially growing cells. RNase protection assays were performed using RiboQuant Apoptosis template sets as described herein. Lanes: 1. MCF-7 cells; 2. MDA-MB-231 cells; 3. HCC 1937 cells; 4. CAMA-1 cells; 5. T-47D cells; 6. AU 562 cells. B. Bcl-2 protein level in breast cancer cell lines was determined by Western blotting. Total protein extracts were prepared from exponentially growing cells. 25 μg of whole cell protein lysate from each sample was subjected to Western blot analysis as described herein. Equal loading is demonstrated by actin protein levels. 1. MCF-7 cells; 2. T-47D cells; 3. HCC 1937 cells; 4. MDA-MB-231 cells; 5. CAMA-1 cells; 6. AU 562 cells.

FIGS. 7A-B. tBid under control of the CMV promoter is the most efficient “killer gene” for breast cancer cells. A. Comparison of tumoricidal activity of a panel of pro-apoptotic genes in breast cancer cells. Cells were plated in duplicates and co-transfected at a ratio of 1:1 with a β-galactosidase reporter plasmid and an empty vector (control) or pro-apoptotic gene constructs. β-galactosidase activity was determined 36 hours later. Cell survival was measured as a percent of β-galactosidase activity in cultures transfected with pro-apoptotic genes relative to vector-transfected control cultures. The data represent an average of at least three independent experiments. B. CAMA-1 cells were transfected with IRES2-EGFP constructs. Unmodified vector and IRES-Luc, in which the luciferase gene served as a surrogate “killer gene” were used as controls to IRES-tBid. Thirty-six hours after transfection, the proportions of GFP positive cells were determined by flow cytometry.

FIGS. 8A-B. Activity of Muc-1, hTERT and survivin promoters in breast cancer cells. A. Evaluation of hTERT, Survivin and Muc-1 promoter activity. Cells in duplicate wells were co-transfected with hTERT, Survivin-Luc or Muc-Luc constructs (in which promoters were driving expression of firefly luciferase) and pRL-TK plasmids at a ratio of 1:1. A dual luciferase assay was performed 36 hour later. The data are presented as a ratio of firefly to Renilla luciferase activity (FL/RL×100) and represent an average of at least three independent experiments. B. Activity of hTERT, Survivin and Muc-1 promoters is significantly lower than that of CMV promoter in breast cancer cells. Transfections and calculations were performed as described above.

FIGS. 9A-B. Expression of hTERT, Survivin and Muc-1 in breast cancer cell lines. A. Expression of Survivin and Muc-1 proteins was determined by Western blot analysis as described herein. Muc-1 is a glycosylated protein with a size ranging from 260 to 400 kD, thus producing a broad band. Lanes: 1. AU 562 cells; 2. CAMA-1 cells; 3. HCC 1937 cells; 4. MCF-7 cells; 5. MDA-MB-231 cells; 6. T-47D cells. B. hTERT mRNA expression was determined by real-time PCR.

FIGS. 10A-B. Efficiency of hTERT-, Survivin- and Muc-1-tBid and their combinations in destroying breast cancer cells. A. Tumoricidal activity of hTERT-, Survivin- or Muc1-guided tBid construct was tested individually. Cells were plated in duplicate wells and transfected with 100 ng of hTERT-, Survivin- or Muc1-guided tBid construct together with 100 ng of β-galactosidase reporter plasmid as described herein. Luciferase under control of the corresponding promoter served as control for each construct. β-galactosidase activity was determined 36 hours later. Cell survival was measured as a percent of β-galactosidase activity in cultures transfected with pro-apoptotic genes relative to luciferase-transfected control cultures. The data represent an average of at least three independent experiments. B. Combinations of different promoters guiding tBid expression decrease cell survival in an additive manner. Cells were transfected with the indicated promoter-tBid combinations as described above. 100 ng of each construct was transfected per well. Combinations of the corresponding promoters, guiding luciferase gene, served as control. Cell survival was estimated as described above.

FIG. 11. Activity of p53 response element in cells with wild type and mutated p53. MCF-7 cells (p53-wt) and MDA-MB-231 cells (p53mut) were co-transfected in duplicate wells with equal amounts of p53C#18 and pRL-TK. A Dual Luciferase assay was performed 36-48 hours later and transfection efficiency was normalized. Results are presented as a ratio to promotorless control.

FIG. 12. Treatment with cisplatin enhances activity of p53-RE in cells with wild type p53. Wild type and p53−/− mouse embryonic fibroblasts (MEF) were transfected with p53C#18 as described above. Increasing concentrations of cisplatin were added at the time of transfection. Luciferase assay was performed 36-48 hours later and transfection efficiency was normalized.

FIG. 13. Cre recombinase under control of p53 response element selectively decreases activity of Lox/Luciferase construct in the presence of wild type p53. Firefly luciferase, as a surrogate “killer gene” under control of tetracyclin response element (TRE) was cloned between two Lox sites to create pLox/LucT. Modified Cre gene was placed under the control of p53 response element 53C#18 (p53mCre). MCF-7 cells with wild type p53 and CAMA-1 and MDA-MB-231 cells carrying p53 mutation were transfected with pLox/LucT alone (control) or with p53mCre, in the presence or absence of exogenous p53. All transfections also included pRL-TK for efficiency normalization. Luciferase activity was measured 48 hours post transfection.

FIG. 14. Tumoricidal activity of tBid under control of CMV promoter in pancreatic cancer cells. MIA-PaCa-2 pancreatic cancer cells were plated in duplicate wells and co-transfected with the promoter-tBid constructs and a β-galactosidase reporter plasmid at a ratio of 1:1. Promoter-Luciferase constructs served as vector control. β-galactosidase activity was determined 36 hours later. Cell survival was measured as a percent of β-galactosidase activity in cultures transfected with tBid relative to vector-transfected control cultures. The data represent an average of at least three independent experiments.

FIG. 15. Activity of tBid under control of hTERT, Survivin and Muc-1 promoters in pancreatic cancer cells. Cells in duplicate wells were co-transfected with hTERT, Survivin-Luc or Muc-Luc constructs (in which promoters were driving expression of firefly luciferase) and pRL-TK plasmid at a ratio of 1:1. A Dual luciferase assay was performed 36 hours later. The data are presented as a ratio of firefly to Renilla luciferase activity (FL/RL×100).

### DETAILED DESCRIPTION OF THE INVENTION

As used herein, “a” or “an” or “the” can mean one or more than one. For example, “a” cell can mean one cell or a plurality of cells.

The present invention is directed to nucleic acids comprising tumor- and/or tissue-specific promoters that direct the expression of apoptosis-inducing (“killer”) genes to produce pro-apoptotic proteins for use in killing tumor cells to treat cancer. Some embodiments of this invention are based on the unexpected discovery that tumor- and/or tissue-specific promoters can be used in combination to drive expression of pro-apoptotic (killer) genes to destroy a greater number of cells within a tumor than would be destroyed by expression of the killer gene(s) under the direction of a single promoter.

Thus, in one embodiment, the present invention provides a nucleic acid comprising a first nucleotide sequence encoding one or more pro-apoptotic proteins, and a second nucleotide sequence encoding one or more tumor- and/or tissue-specific promoters operably linked and directing expression of the first nucleotide sequence. By “one or more” is meant one, two (or more), three (or more), four (or more), five (or more), six (or more), seven (or more), eight (or more), nine (or more), ten (or more), 11 (or more), 12 (or more), 13 (or more), 14 (or more), 15 (or more), 20 (or more), 25 or more), 30 (or more), etc., or more proteins and/or promoters of this invention in any combination.

In other embodiments of this invention, it is contemplated that the nucleic acid of this invention can be used to induce apoptosis in tumor cells that lack a functional p53 protein.

Mutations that disrupt p53 function are the most frequently encountered mutations in human cancer. Thus selective elimination of cells bearing these mutations can have a profound impact on the disease. Because this is a “loss of function” mutation, the usual strategy employed in transcriptional targeting, namely utilization of a promoter region or a DNA binding element which has higher activity in neoplastic as opposed to normal cells, cannot be applied. Instead, transactivating activity of wild type p53 protein, which is present in normal cells, can be used to prevent a “killer gene” from functioning. This approach employs a modified site-specific phage recombinase, Cre, which excises DNA between LoxP sites. Targeting of p53-negative cells involves placing the Cre gene under the control of the p53 response element. Consequently, expression of Cre recombinase will be limited to cells with intact p53 transcriptional function. A “killer gene” placed between Lox sites will be excised in p53-expressing cells. In neoplastic cells with a lack of functional p53, Cre will not be produced and the “killer gene” will remain active.

Thus, the present invention also provides a nucleic acid comprising a nucleotide sequence encoding a pro-apoptotic protein positioned between LoxP sequences and functionally (operably) linked to a nucleotide sequence encoding Cre recombinase under the control of a p53 response element. This nucleic acid can be present as a separate construct or as part of a construct of this invention comprising a first nucleotide sequence encoding one or more pro-apoptotic proteins, and a second nucleotide sequence encoding one or more tumor- and/or tissue-specific promoters operably linked to and directing expression of the first nucleotide sequence.

The tumor-specific and/or tissue-specific promoter of this invention can be, but is not limited to, survivin promoter, MUC-1 promoter, htert promoter, CEA promoter, PSA promoter, alpha-fetoprotein (AFP) promoter, and a promoter that directs expression of a nucleotide sequence encoding any of the cancer and/or tissue-specific antigens described herein, as well as any cancer or tissue-specific antigen now known or later identified, and any combination thereof.

The hTERT gene encodes a catalytic subunit of human telomerase. Telomerase activity is detected in a majority of human tumors and is absent from normal adult tissues with the exception of a fraction of actively proliferating hematopoietic and germ cells.

Survivin, a member of the LAP anti-apoptotic gene family, is highly and consistently overexpressed in neoplastic cells of diverse origin. Its overexpression correlates with poor prognosis in a majority of cancer types investigated. Survivin is undetectable in normal adult tissues except small portions of CD34+ hematopoietic cells, T-lymphocytes and colonic mucosa DF3/Muc-1 is a gene normally expressed in small subpopulations of epithelial cells from various tissues. It is highly overexpressed in 90-100% of pancreatic cancer as well as in other epithelial tumors, including gastric, colon and breast.

A cancer antigen of this invention can include, but is not limited to, for example, HER2/neu and BRCA1 antigens for breast cancer, MART-1/MelanA, gp100, tyrosinase, TRP-1, TRP-2, NY-ESO-1, CDK-4, β-catenin, MUM-1, Caspase-8, KIAA0205, HPVE7, SART-1, PRAME, and p15 antigens, members of the MAGE family, the BAGE family (such as BAGE-1), the DAGE/PRAME family (such as DAGE-1), the GAGE family, the RAGE family (such as RAGE-1), the SMAGE family, NAG, TAG-72, CA125, mutated proto-oncogenes such as p21ras, mutated tumor suppressor genes such as p53, tumor associated viral antigens (e.g., HPV16 E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE-4 and MAGE-11. Members of the GAGE family include, but are not limited to, GAGE-1, GAGE-6. See, e.g., review by Van den Eynde and van der Bruggen (1997) in Curr. Opin. Immunol. 9: 684-693, Sahin et al. (1997) in Curr. Opin. Immunol. 9: 709-716, and Shawler et al. (1997), the entire contents of which are incorporated by reference herein for their teachings of cancer antigens.

The cancer antigen can also be, but is not limited to, human epithelial cell mucin (e.g., MUC-1; a 20 amino acid core repeat for Muc-1 glycoprotein), MUC-2, MUC-3, MUC-18, the Ha-ras oncogene product, carcino-embryonic antigen (CEA), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), alpha-fetoprotein (AFP), Bcl-xL, Mcl-1, Bcl-2, survivin, htert, probasin, ARR2PB, CO17-1A, GA733, gp72, p53, the ras oncogene product, β-HCG, gp43, HSP-70, p17 mel, HSP-70, gp43, HMW, HOJ-1, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3, 19-9, 549, 195, squamous cell carcinoma antigen (SCCA), ovarian cancer antigen (OCA), pancreas cancer associated antigen (PaA), mutant K-ras proteins, mutant p53, and chimeric protein P210_{BCR-ABL} and tumor associated viral antigens (e.g., HPV16 E7). Also included in this invention are other substances that are overexpressed in cancer cells as compared to normal cells, such as, for example, midkine and cyclooxygenase promoters for pancreatic and colon cancer.

The cancer antigen of this invention can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the cancer antigen of this invention can be a single chain antibody (scFv), comprising linked V_{H}, and V_{L} domains, which retains the conformation and specific binding activity of the native idiotype of the antibody.

The present invention is in no way limited to the cancer antigens listed herein. Other cancer antigens be identified, isolated and cloned by methods known in the art such as those disclosed in U.S. Pat. No. 4,514,506, the entire contents of which are incorporated by reference herein.

In some embodiments, the nucleic acid sequence of this invention can further comprise one or more pro-apoptotic proteins that function as a pro-apoptotic mediator (i.e., it enhances or otherwise positively mediates the effect of the killer gene and/or its protein product). In other embodiments, the nucleic acid sequence of this invention can be present in a composition with a separate nucleic acid sequence encoding a pro-apoptotic mediator and in yet other embodiments, the nucleic acid sequence encoding a pro-apoptotic mediator can be separate from the nucleic acid of this invention and separate from a composition comprising the nucleic acid of this invention.

It is also understood that the promoters and killer genes and pro-apoptotic mediators of this invention can be present on a single construct, on separate constructs, or any combination thereof. These constructs can be present in a single composition or in separate compositions of this invention in any combination. For example, a composition of this invention can comprise a nucleic acid comprising one or more promoters of this invention, one or more killer genes of this invention and/or one or more pro-apoptotic mediators of this invention. In other embodiments, a composition of this invention can comprise a first nucleic acid comprising a first promoter and a first killer gene, a second nucleic acid comprising a second promoter and a second killer gene, etc., such that the composition comprises a multiplicity of promoters and killer genes present in the composition on separate nucleic acids. Such a composition can further comprise a nucleic acid encoding a pro-apoptotic mediator, which can be a separate nucleic acid or which can be included with a nucleic acid of the composition comprising a promoter and killer gene. In some embodiments, the pro-apoptotic mediator can be present in a composition of this invention as a protein and/or peptide (e.g., TRAIL, Smac).

The pro-apoptotic protein of this invention, which can be encoded by a killer gene and which can also be a pro-apoptotic mediator, can be, but it not limited to tBid (amino acids 60-195, base pairs +322+732 of the Bid gene; formed from cleavage of inactive precursor, Bid, by caspase 8), Bax, Death Receptor 4 (DR4), Death Receptor 5 (DR5), HAPR, Bak, Bad, Bim, Bid, Bik, Harakiri, Noxa, bcl-Xs, other pro-apoptotic members of the bcl-2 family, Smac, caspase 3, caspase 8, caspase 9, Fas receptor, Fas ligand, tumor necrosis factor (TNF) receptor, TNF, TNF-related apoptosis inducing ligand (TRAIL), FasL, Noxa, PUMA, p53AlP1, TGF-0, Granzyme A, Granzyme B, Apafl, TRADD, FADD and/or a biologically active fragment of any of the above, as well as any other pro-apoptotic protein now known or later identified.

As one example, a particular embodiment of this invention can be a nucleic acid comprising a nucleotide sequence encoding DR4 and a nucleotide sequence encoding a promoter selected from the group consisting of htert, survivin, Muc-1, and any combination thereof. This killer gene and these promoters can be present on a single nucleic acid construct or they can be present as separate nucleic acid constructs (e.g., htert/DR4, survivin/DR4 and Muc-1/DR4). The nucleic acid of this embodiment can further comprise a nucleotide sequence encoding Bax and/or FADD and/or Smac and/or TRAIL as pro-apoptotic mediators. Genes encoding these pro-apoptotic mediators can also be present on separate constructs. Alternatively, these pro-apoptotic mediators can be present as proteins.

The term “nucleic acid” as used herein refers to single- or double-stranded molecules which can be DNA, comprised of the nucleotide bases A, T, C and G, or RNA, comprised of the bases A, U (substitutes for T), C, and G. The nucleic acid can represent a coding strand or its complement. Nucleic acids can be identical in sequence to the sequence that is naturally occurring or can include alternative codons, which encode the same amino acid as that which is found in the naturally occurring sequence. Furthermore, nucleic acids may include codons that provide conservative substitutions of amino acids as are well known in the art. The nucleic acids of this invention can also comprise any nucleotide analogs and/or derivatives as are well known in the art.

As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by well-known techniques such as cell lysis followed by phenol plus chloroform extraction, followed by ethanol precipitation of the nucleic acids.

The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the polypeptide encoded by the nucleic acid are maintained.

The nucleic acid of this invention can be part of a recombinant nucleic acid construct comprising any combination of restriction sites and/or functional elements as are well known in the art that facilitate molecular cloning and other recombinant DNA manipulations. Thus, the present invention further provides a recombinant nucleic acid construct comprising a nucleic acid of this invention.

The nucleic acid of this invention can be in a cell, which can be a cell expressing the nucleic acid of this invention. In addition, the vector of this invention can be in a cell, which can be a cell expressing the nucleic acid of the vector in the cell. The nucleic acid of this invention can also include, for example, antibiotic resistance markers and/or other selectable and/or screenable-markers as are known in the art, origins of replication and/or expression control sequences, such as, for example, a promoter (constitutive or inducible), an enhancer and necessary information processing sites, such as initiation signals, ribosome binding sites, RNA splice sites, multiple cloning sites, polyadenylation sites and transcriptional terminator sequences. The nucleic acid of this invention can also comprise one or more internal ribosome binding sites (IRES) for expression of more than one coding sequence from the same construct.

A nucleic acid encoding a polypeptide of this invention can readily be determined based upon the genetic code for the amino acid sequence of the selected polypeptide and many nucleic acids will encode any selected polypeptide, based upon the redundancy of the genetic code. Modifications in the nucleic acid sequence encoding the polypeptide are also contemplated. Modifications that can be useful are modifications to the sequences controlling expression of the polypeptide to make production of the polypeptide inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

For example, the nucleic acids and vectors of this invention can be introduced into cells via any gene transfer mechanism, such as, for example, virus-mediated gene delivery, calcium phosphate mediated gene delivery, lipofection, electroporation, uptake by cells via endocytosis, microinjection or proteoliposomes.

The present invention further provides a vector comprising a nucleic acid of this invention. The vector of this invention can be any type of vector that facilitates delivery of nucleic acid to a cell. A vector of this invention can be a nucleic acid vector such as a plasmid, cosmid, virus, and/or an artificial chromosome. The vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols.

In some embodiments, the expression vector can comprise, for example, viral nucleic acid including, but not limited to, nucleic acid from vaccinia virus, adenovirus, lentivirus/retrovirus, alphavirus, herpesvirus, vaccinia virus, polyoma virus, hybrid adeno/adeno-associated virus and/or adeno-associated virus (AAV; see for example, Owens (2002) “Second generation adeno-associated virus type 2-based gene therapy systems with the potential for preferential integration into AAVS1” Curr. Gene Ther. 2:145-159, the entire contents of which are incorporated herein by reference for teachings of AAV vectors), as well as any other viral vector now known or later identified according to methods well known in the art.

In yet other embodiments, the vector of this invention can be any vehicle for delivery of nucleic acid into a cell that is lipid-, peptide-, and/or protein-based. For example, the nucleic acid or vector of this invention can also be in a liposome (e.g., FuGene6; VDL liposomes) or a delivery vehicle, which can be taken up by a cell via receptor-mediated or other type of endocytosis. The vectors, liposomes and other delivery vehicles of this invention can further comprise molecules on the surface that allow for specific cell targeting and binding, as are well known in the art.

As one example, the nucleic acid and/or vector of this invention can be in a liposome developed in the Vector Development Laboratory (VDL) of Baylor College of Medicine. According to the manufacturer, “These liposomes and nucleic acid liposome complexes have extended half life in the circulation, are stable in serum, have broad biodistribution, efficiently encapsulate all types of nucleic acids, are targetable to specific organs and cell types, are able to penetrate through tight barriers in several organs, and have been optimized for nucleic acid:lipid ratio and colloidal suspension in vivo.”

Administration of the nucleic acids of this invention can be achieved by any of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and cell populations by using the targeting characteristics of the carrier, which would be well known to the skilled artisan.

Vectors employed in the methods of this invention can be any nucleotide construct used to deliver nucleic acid into cells, e.g., a plasmid or viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). The recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff., Nature 352:815-818, (1991).

In further embodiments, the nucleic acids of this invention as described above can be combined in an administration protocol with a chemotherapeutic agent, an immunotherapeutic agent, a surgical procedure and/or radiation. For example, it has been shown that genotoxic stress, including chemotherapy medications, UV and/or gamma radiation enhances apoptosis induced by pro-apoptotic “killer genes.” It also enhances transduction efficiency and increases transgene expression several fold (Sanlioglu et al. (1999) “Two independent molecular pathways for recombinant adeno-associated virus genome conversion occur after UV-C and E4orf6 augmentation of transduction” Hum. Gene Ther. 10:591-602; Peng et al., (2000) “Transduction of hepatocellular carcinoma (HCC) using recombinant adeno-associated virus (rAAV): in vitro and in vivo effects of genotoxic agents” J. Hepatol. 32:975-985, the entire contents of which are incorporated by reference herein for teachings of augmentation of transduction and apoptotic activity of transduced killer genes).

A chemotherapeutic agent of this invention can be, but is not limited to, irinotecan, gemcytobine, cisplatin, carboplatin, procarbazine, mechlorethamine, cyclophsphamide, camptothecin, ifosfamide, melphalan, chlorambucil, busulfran, nitrosurea, dactinomycin, daunorubicin, doxorubicin, bleomycin, plicomycin, mitomycin, etoposide, tamoxifen, raloxifene, estrogen receptor binding agents, taxol, paclitaxol, gemcitabien, navelbine, farnesyl-protein transferase inhibitors, transplatinum, 5-fluorouracil, floxuridine, mutamycin, vincristin, vinblastin, methotrexate, antibodies, small molecules (e.g., Herceptin, tyrosine kinase inhibitors, signal transduction inhibitors, etc.) as well as any analogue or derivative of a chemotherapeutic agent of this invention.

Radiation employed in this invention can be, but is not limited to, ultraviolet irradiation, gamma radiation, X rays, microwaves, delivery of radioisotopes to tumor cells The radiation dosage employed will vary on the basis of the subject to be treated, the type and location of the tumor to be treated, the other agents with which it is combined in a therapy regimen, etc., according to protocols standard in the art.

An immunotherapeutic agent of this invention can be, but is not limited to antibodies, anti-idiotype antibodies, antigens and immunostimulatory cytokines, (e.g., GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), as well as any other immunotherapeutic and/or immunostimulatory substance now known or later identified.

In some embodiments of this invention, the composition of this invention can include an adjuvant. As used herein, “adjuvant” describes a substance, which can be any immunomodulating substance that can enhance, improve or otherwise modulate an immune response in a subject without deleterious effect on the subject.

An adjuvant of this invention can be, but is not limited to, for example, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, a composition of this invention can include an adjuvant by comprising a nucleic acid comprising a nucleotide sequence encoding an adjuvant function, such as CpG sequences. Such CpG sequences, or motifs, are well known in the art.

An adjuvant of this invention, such as, for example, an immunostimulatory cytokine, can be administered before, concurrent with, and/or within a few hours, several hours, and/or 1, 2, 3, 4, 5, 6, 7, 8, 9, and/or 10 days before or after the administration of a composition of this invention to a subject.

A chemotherapeutic agent and/or immunotherapeutic agent of this invention can be included in a composition of this invention with a nucleic acid and/or vector of this invention so that the chemotherapeutic agent and/or immunotherapeutic agent is administered to a subject simultaneously with nucleic acid and/or vector. A chemotherapeutic agent and/or immunotherapeutic agent of this invention can also be in a composition without a nucleic acid and/or vector of this invention so that the chemotherapeutic agent and/or immunotherapeutic agent can be administered either before, simultaneously with, and/or after administration of the nucleic acid and/or vector of this invention to a subject. If irradiation and/or a surgical procedure is employed in the methods of this invention, it can be administered to a subject either before, simultaneously with, and/or after administration of the nucleic acid and/or vector and/or chemotherapeutic agent and/or immunotherapeutic agent of this invention.

When administered to a subject in the same treatment protocol (either simultaneously or at separate time points in any sequence), the nucleic acid and/or vector of this invention and the chemotherapeutic agent and/or immunotherapeutic agent of this invention can be administered to the subject in amounts that produce a ratio of nucleic acid/vector to chemotherapeutic agent and/or immunotherapeutic agent of 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90 and/or 1:100 or more.

Also, when administered to a subject in the same treatment protocol (either simultaneously or at separate time points in any sequence), the nucleic acid and/or vector of this invention comprising one or more tumor-specific promoters and one or more nucleotide sequences encoding a pro-apoptotic protein (nucleic acid 1) and the nucleic acid and/or vector of this invention comprising one or more nucleotide sequences encoding a pro-apoptotic protein functioning as a pro-apoptotic mediator (nucleic acid 2) can be administered to the subject in amounts that produce a ratio of nucleic acid 1 to nucleic acid 2 of 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90 and/or 1:100 or more.

As indicated above, the nucleic acids, vectors and/or chemotherapeutic agents of this invention can be present in a composition comprising a pharmaceutically acceptable carrier Thus, pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. The carrier can be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, vaginal, topical, inhalation (e.g., via an aerosol) buccal (e.g., sub-lingual), vaginal, parenteral (e.g., subcutaneous, subconjunctival, intravesicular, intramuscular, intradermal, intraarticular, intrapleural, intratracheal, intraperitoneal, intracerebral, intraarterial, intracranial, intraocular, intratumoral, intravenous, etc.), topical (i.e., both skin and mucosal surfaces, including airway surfaces) and/or transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

In particular embodiments, more than one administration (e.g., two, three, four or more administrations) may be employed to achieve the desired level of gene expression.

In some embodiments of the invention, the nucleic acid or vector is administered to the CNS (e.g., to the brain or to the eye). The vector can be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes cortex, basal ganglia, hippocampus and amygdala), limbic system, neocortex, corpus striatum, cerebrum, and inferior colliculus. The vector can also be administered to different regions of the eye such as the retina, cornea or optic nerve.

The nucleic acid or vector can be delivered into the cerebrospinal fluid (e.g., by lumbar puncture) for more disperse administration. The nucleic acid or vector can further be administered intravascularly to the CNS in situations in which the blood-brain barrier has been perturbed (e.g., brain tumor or cerebral infarct).

The nucleic acid or vector can be administered to the desired region(s) of the CNS by any route known in the art, including but not limited to, intrathecal, intra-ocular, intracerebral, intraventricular, intranasal, intra-aural, intra-ocular (e.g., intra-vitreous, sub-retinal, anterior chamber) and peri-ocular (e.g., sub-Tenon's region) delivery.

Typically, the nucleic acid or vector is administered in a liquid formulation by direct injection (e.g., stereotactic injection) to the desired region or compartment in the CNS. In other embodiments, the nucleic acid or vector is provided by topical application to the desired region or by intra-nasal administration of an aerosol formulation. Administration to the eye or into the ear, can be by topical application of liquid droplets. As a further alternative, the nucleic acid or vector can be administered as a solid, slow-release formulation. For example, controlled release of parvovirus and AAV vectors is described by international patent publication WO 01/91803.

In other embodiments, a nucleotide sequence of interest is administered to the liver of the subject. Administration to the liver may be achieved by any method known in the art, including, but not limited to intravenous administration, intraportal administration, intrabiliary administration, intra-arterial administration, and direct injection into the liver parenchyma.

The nucleic acids and vectors disclosed herein may be administered to the lungs of a subject by any suitable means, but are preferably administered by administering an aerosol suspension of respirable particles comprised of the delivery vectors, which the subject inhales. The respirable particles may be liquid or solid. Aerosols of liquid particles comprising the delivery vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the delivery vectors may likewise be produced with any solid particulate medicament aerosol generator, by techniques known in the pharmaceutical art.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, scream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bistris buffer (pH 6) or ethanol/water and can contain from 0.1 to 0.2M active ingredient.

Thus the present invention also provides a method of treating a cancer in a subject, comprising administering to the subject an effective amount of a nucleic acid, a vector and/or a composition of this invention to the subject, thereby treating the cancer in the subject. The nucleic acid of this invention can comprise one or more tumor- and/or tissue-specific promoters that direct expression of one or more killer genes in any combination. Such a combination of promoters and killer genes can be present on a single nucleic acid construct for simultaneous administration and/or on multiple nucleic acid constructs for simultaneous and/or sequential administration of the various promoter/killer gene combinations to the subject.

The methods of this invention can also include the steps of administering to the subject an effective amount of one or more pro-apoptotic mediators, one or more chemotherapeutic and/or immunotherapeutic agents, surgical procedures and/or radiation to the subject, either before, simultaneously with, and/or after administration of the nucleic acid, vector and/or composition to the subject.

It is also contemplated in this invention that a nucleic acid can be customized to treat the cancer of a particular subject. Such customization involves identifying a tumor- and/or tissue-specific protein and/or antigen that is overexpressed in tumor cells of the subject and/or identifying a tumor- and/or tissue specific promoter having high activity (i.e., a level of activity greater than a level of activity detectable by similar means in a normal cell) and employing one or more promoters of such identified proteins or antigens, and/or one or more promoters identified as having high activity, in a nucleic acid of this invention comprising one or more pro-apoptotic genes.

Thus, in a further embodiment, the present invention provides a method of treating cancer in a subject, comprising: a) identifying an overexpressed tumor-specific and/or tissue-specific protein in tumor cells of the subject and/or identifying a tumor- and/or tissue-specific promoter having high activity in tumor cells of the subject; b) producing a first nucleic acid comprising a nucleotide sequence comprising one or more promoters of the overexpressed tumor- and/or tissue-specific proteins identified in (a) and/or a tumor- and/or tissue-specific promoter having high activity identified in step (a), and a second nucleotide sequence encoding one or more pro-apoptotic proteins; and c) administering an effective amount of the nucleic acid of step (b) to the subject, thereby treating cancer in the subject.

The method of treating cancer in a subject by producing a custom nucleic acid can further include the steps of administering an effective amount of one or more pro-apoptotic mediators (either as a nucleic acid or a protein), one or more chemotherapeutic and/or immunotherapeutic agents, and/or surgical procedures and/or radiation to the subject, either before, simultaneously with, and/or after administration of the nucleic acid of step (b).

The identification of overexpressed tumor-specific proteins in tumor cells of a subject and the identification of tumor-specific promoters having high activity in a subject's tumor cells can be carried our according to methods well known in the art and as described herein.

The present invention further provides methods of treating cancer in a subject that has tumor cells that are resistant to apoptosis. These methods involve the identification of a pro-survival gene and/or other gene that is overexpressed in apoptosis-resistant tumor cells of the subject and that is responsible for the resistance to apoptosis. Once such a gene is identified, the compositions of this invention are administered to the subject, in combination (either before, simultaneously with and/or after) with a nucleic acid encoding an antisense sequence for silencing the apoptosis-resistance inducing gene. In another embodiment, the compositions of this invention can be administered to the subject in combination with a substance (e.g., a nucleic acid encoding a suppressor protein, a nucleic acid encoding a dominant-negative mutant, antisense DNA, siRNA, ribozymes, etc.) that acts to suppress the effect of the apoptosis-resistance inducing gene.

A subject of this invention is any subject that is susceptible to cancer and who may be in need of and/or who could acquire a beneficial effect from the treatment methods of this invention (e.g., a subject suspected of having or diagnosed with cancer). The subject of this invention can be, for example, avian or mammalian and in some embodiments, is a human.

Efficacy of the treatment methods of this invention can be determined according to well known protocols for determining the outcome of a cancer treatment. For example, tumor size and quantity can be monitored to identify a decrease in the size and/or number of tumors, and/or assays of serum can be conducted to identify a decrease in the amount of cancer antigen present in the serum of a subject. Other determinants of efficacy of treatment, include, for example, overall survival, disease-free survival, time to progression and/or quality of life, as are well known in the art.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

The cancer to be treated by the methods of this invention can be, but is not limited to, B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, adenocarcinoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, uterine cancer, cervical cancer, gastrointestinal cancer, and any other cancer now known or later identified (see, e.g., Rosenberg (1996) Ann. Rev. Med. 47:481-491, the entire contents of which are incorporated by reference herein).

“Effective amount” refers to an amount of a compound or composition that is sufficient to produce a desired effect, which can be a therapeutic effect. The effective amount will vary with the age, general condition of the subject, the severity of the condition being treated, the particular biologically active agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

In general, a dosage range from about 0.001 μg/kg to about 500 mg/kg of a composition of this invention, including any dosage amount or dosage sub-range within this range, will have therapeutic efficacy, with all weights being calculated based upon the weight of the composition.

When the composition of this invention is to be administered to a subject as a viral vector, a suitable dosage range can be determined for that viral vector according to standard protocols. Dosages of viral vectors to be administered to a subject will depend upon the mode of administration, the disease or condition to be treated, the individual subject's condition, the particular virus vector, and the nucleic acid to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵ transducing units or more, preferably about 10⁸-10¹³ transducing units, yet more preferably 10⁹ to 10¹² transducing units.

As one example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 10¹² pfu per injection (Crystal (1997) “Phase I study of direct administration of a replication deficient adenovirus vector containing E. coli cytosine deaminase gene to metastatic colon carcinoma of the liver in association with the oral administration of the pro-drug 5-fluorocytosine” Human Gene Therapy 8:985-1001; Alvarez and Curiel (1997) “A phase I study of recombinant adenovirus vector-mediated delivery of an anti-erbB-2 single chain (sFv) antibody gene from previously treated ovarian and extraovarian cancer patients” Hum. Gene Ther. 8:229-242; the entire contents of which are incorporated by reference herein for teachings of administration of viral vectors).

The frequency of administration of a composition of this invention can be as frequent as necessary to impart the desired therapeutic effect. For example, the composition(s) can be administered one, two, three, four or more times per day, one, two, three, four or more times a week, one, two, three, four or more times a month, one, two, three or four times a year, etc., as necessary to control the condition. The different compositions described herein can be administered simultaneously and/or sequentially in any order, which can be repeated, reversed and/or otherwise varied. Intervals between sequential administrations of different compounds can be optimized according to methods known in the art such that an advantageously combined effect is achieved. The amount and frequency of administration of the composition(s) of this invention will vary depending on the particular condition being treated and the desired therapeutic effect.

The compositions of this invention can be administered to a cell of a subject in vivo or ex vivo. For administration to a cell of the subject in vivo, as well as for administration to the subject, the compositions of this invention can be administered for example as noted above, orally, parenterally (e.g., intravenously or intra-arterially), by intramuscular injection, intradermally (e.g., by gene gun), by intraperitoneal injection, subcutaneous injection, transdermally, extracorporeally, topically, intratumorally or the like.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

The present invention additionally provides kits comprising the nucleic acid and/or vectors and/or compositions of this invention, with or without pro-apoptotic mediators and/or chemotherapeutic and/or immunotherapeutic agents, along with appropriate buffers, diluents, vessels and/or devices, etc. for measuring a specific amount and for administering the compositions to a subject of this invention.

The present invention further provides methods and compositions of producing viral vectors comprising the nucleic acids of this invention. In particular, it is contemplated that expression of killer genes in packaging cell lines during virus particle production would be toxic to the packaging cell, thereby reducing viral titers. Thus, in order to protect the packaging cell from the toxic effects of the gene products of the killer genes, the expression of the killer gene can be silenced with siRNA. This can be accomplished by stable transformation of the packaging cells with an siRNA DNA vector and/or an antisense sequence specific for the killer gene(s) to be packaged (e.g., pGE vector from Stratagene or pSuper vector from OligoEngine). Alternatively, pGE-si-killer gene and/or antisense sequence could be included in a co-transfection together with recombinant pAAV expression plasmids pHelper and pAAV-RC in an AAV packaging system.

The use of siRNA is a sequence-specific posttranscriptional gene silencing technique, in which expression of the target gene is down regulated by introduction of homologous double-stranded RNA (dsRNA). While long stretches of dsRNA produce non-specific cytotoxicity, short fragments of 19-30 nt with 2-nt 3′ overhang, known as short interfering RNAs (siRNA), are non-toxic, highly efficient and specific. In certain embodiments, to achieve specific silencing of the “killer genes,” the plasmid vector, pSUPER, specifically designed for vector-based siRNA techniques is employed. For each killer gene to be silenced in the packaging cell, an oligonucleotide corresponding to the target transcript is cloned downstream of the polymerase III H1-RNA promoter. This oligonucleotide is designed to contain a 19-nt sequence derived from target RNA separated by a 9-nt spacer from the reverse complement of the same sequence. The resulting transcript folds back on itself, producing double-stranded siRNA. Selection of the target sequence for oligonucleotide production is performed with a computer program designed to identify mRNA sequences, optimized for siRNA technique.

In some embodiments, the actual silencing can be accomplished in two ways. First, the pSUPER (or similar) siRNA construct can be included in a construct in co-transfection performed for viral stock production, together with recombinant pAAV expression plasmids, pHelper and pAAV-RC. If this approach fails to protect packaging cell line from apoptosis, the packaging cells can be stably transformed with the pSUPER-siRNA vector with subsequent selection of killer gene-resistant clones.

In other embodiments, a construct comprising a bcl-2 gene under the direction of a highly active (constitutive or inducible) promoter can be introduced into the packaging cell, resulting in high levels of bcl-2, which would protect the packaging cell from apoptosis induced by expression of the killer gene(s) to be packaged. In further embodiments, an antisense sequence specific for the killer gene can be added to the packaging cell to silence its expression. Such antisense sequences can be produced according to methods well known in the art. These strategies to minimize the toxic effect of the killer gene in packaging cells can be employed singly or in any combination.

Thus, the present invention further provides a method of producing a viral vector comprising a nucleic acid of this invention comprising a nucleotide sequence encoding a pro-apoptotic protein, comprising introducing into a packaging cell an siRNA construct that targets the nucleotide sequence encoding a pro-apoptotic protein, and/or a nucleic acid encoding bcl-2 operably linked to an active promoter, and/or an antisense sequence specific for the killer gene, either prior to, or simultaneously with introducing into the packaging cell the nucleic acid comprising a nucleotide sequence encoding a pro-apoptotic protein.

Further provided is a packaging cell comprising the components described above that function to silence killer genes and/or minimize apoptotic effects of the gene products of killer genes. As indicated above, these elements can be present in the packaging cell transiently or the packaging cell can be stably transformed with nucleic acid expressing these elements under the control of a constitutive and/or inducible promoter.

The nucleic acids of this invention can be packaged into viral particles according to standard protocols for producing viral vectors. As one example, for packaging into an AAV vector, the following protocol can be employed. Recombinant AAV expression plasmids are produced with the components of an AAV helper-free system (Stratagene) by cloning a killer gene into the multiple cloning site of pAAV-MCS and replacing the CMV promoter with a tumor-specific and/or tissue-specific promoter of this invention. These expression plasmids are then transfected into AAV-293 packaging cells together with the pAAV-RC (carrying AAV capsid and replication proteins) and phelper (harboring adenoviral E2A, E4 and VA genes) by calcium phosphate precipitation. An siRNA construct, bcl-2 construct and/or antisense sequence can also be added at this time to silence the expression of the killer gene and/or minimize the apoptotic effect of the expressed killer gene product in the packaging cells. The packaging cell line stably expresses adenoviral E1 gene, which completes the requirements for production of virus particles. Approximately three days post-transfection, supernatants and cell lysates prepared by freeze-thaw cycles are collected. Titration of viral stocks is by dot-blot-hybridization. Further concentration, if needed, is done by CsCl centrifugation.

In other embodiments, the vectors of this invention can be produced by in vitro methods (e.g., cell-free methods) that are known in the art, thereby avoiding the issue of toxicity in a packaging cell.

### EXAMPLES

### Example 1 DR4 Studies

Cell lines and reagents. Human breast cancer cell lines MCF-7, CAMA-1, HCC 1937, MDA-MB-231, T-47D and AU 562 were obtained from American Type Culture Collection (ATCC). Cells were maintained in RPMI 1640 (T47D, AU-562 and HCC 1937), A-MEM (MCF-7 and CAMA-1) or Leibovitz's-15 (MDA-MB-231) medium with 10% FBS, penicillin (100 U/ml) and streptomycin (100 μg/ml) at 37° C. Except for MDA-MB-231, which did not require CO₂ supplementation, all cell lines were grown in humidified atmosphere with 5% CO₂. These six cell lines differ in expression of genes relevant to pathogenesis and prognosis of disease, such as the estrogen receptor (ER) expressed in MCF-7, T-47D and CAMA-1 cells; Her2/neu, expressed in T-47D and AU 562 cells and possibly in CAMA-1 cells; mutated p53 tumor suppressor, detected in all of these cell lines except MCF-7; and BRCA1 mutations, expressed in HCC 1937 cells. There is also a moderate c-myc amplification in CAMA-1 cells and MCF-7 cells are bcl-2 positive and deficient in caspase 3. This genetically diverse panel represents frequently encountered variants of breast cancer.

Reagents used in the study were obtained from the following companies: FuGENE 6 Transfection Reagent—Roche Applied Science; Dual-Luciferase Reporter Assay System—Promega; Luminescent beta-gal detection kit—BD Clontech; soluble TRAIL (residues 114-281 of the soluble domain of human TRAIL, expressed in E. coli.)—BIOMOL Research Laboratories, Inc; CellTiter 96 Non-Radioactive Cell Proliferation Assay (e.g., MTS assay)—Promega; ApoAlertm Annexin V-PE Apoptosis kit—Clontech; hApo Multi-Probe Template Sets—BD Biosciences; Antibodies to DR4, DR5, FLIP and TRAIL—QED Bioscience Inc; Donkey Anti-Rabbit IgG peroxidase-labeled antibody and ECL™ Western Blotting Detection Reagents—Amersham Biosciences.

Plasmids and cloning. pRL-TK and pSV40-beta-galactosidase were purchased from Promega. DR4 cDNA was obtained from ATCC Mammalian Gene Collection (BC012866), cut out of pCMV-Sport6 on EcoRI/ApoI and cloned into EcoRI sites of pcDNA3.1 and IRES2-EGFP to produce CMV-DR4 and IRES-DR4, respectively. pp 76, containing hTERT promoter—1375 in pGL3 Basic vector was kindly provided by Dr. Kyo. It was cut on Mlu I/Pst I, blunt-ended and re-closed to delete Xba I site. Luciferase activity of the resultant construct, tested in a panel of breast cancer cell lines, did not differ from that of pp 76. pp 76 was then restricted on Xba I/Hind III to delete luciferase gene and DR4, cut out of pcDNA3.1 on Xba I/Hind III, was introduced in its place to produce hTERT-DR4. To produce CMV promoter-guided luciferase, luciferase gene was cut out of pGL3-Basic on Hind III/Xba I, cloned into Hind III/Xba I site of pcDNA3.1 and called pcD-Luc.

Transfections and Reporter assays. Transient transfections were performed with FuGENE 6 Transfection Reagent per manufacturer's protocol. Briefly, 5×10⁴ cells per well were plated in 24 well plates and incubated overnight. For promoter activity studies, hTERT- or CMV-firefly luciferase promoter construct was co-transfected with pRL-TK (Promega) at a ratio of 1:1 into duplicate wells. Cells were harvested 36 hours later and dual luciferase assay was performed using Promega Dual-Luciferase Reporter Assay System per manufacturer's protocol. Results are presented as a ratio of Firefly/Renilla luciferase activity. For cell killing experiments, 5×10⁴ cells in duplicate wells were co-transfected with a DR4-containing construct or a corresponding vector together with the pSV40-beta-galactosidase reporter gene at a ration of 1:1. When indicated, soluble TRAIL was added to cultures at the time of transfection. Beta-galactosidase activity was measured with a luminescent beta-gal detection kit 36 hours later. Relative cell survival was estimated as a percent of beta-galactosidase reporter activity in DR4 vs vector-transfected cultures.

MTS cytotoxicity assay. Cells were plated 2×10⁴ per well in triplicate wells and incubated overnight. Serial dilutions of soluble TRAIL in Hank's balanced salt solution (HBSS) were added to the wells and cells were incubated for an additional 36 hours. Cytotoxicity was assessed using a CellTiter 96 Non-Radioactive Cell Proliferation Assay and medium background was subtracted. Results are presented as percent of HBSS-treated control.

Annexin V determination. 1×10⁶ cells were plated per 60 mm dish and cultured overnight. Cells were than left untreated or treated with soluble TRAIL (100 ng/ml) and incubated for additional 36 hours. Cells were than trypsinized and washed with 1× Binding Buffer. For each condition, half of the cell suspension was left untreated and another half was incubated for 15 minutes at room temperature in the dark with Annexin V. Immediately after staining, cells were analyzed by flow cytometry.

Western Blotting. Exponentially growing cells were lysed in lysis buffer (62.5 mM Tris-HCL, pH 6.8, 2% SDS, 1 mM phenylmethylsulfonyl fluoride (PMSF). Cell lysates were sonicated and protein concentrations were determined using Bio-Rad protein assay reagent. 25 μg of whole cell protein lysate from each sample was diluted in 25 μl of SDS loading buffer (4% SDS, 2% Glycerol, 0.01% Bromphenol blue and 125 mM Tris-HCL, pH 6.8), incubated 5 minutes at 98° C., placed for 5 minutes on ice and spun down at maximal speed in a microcentrifige at room temperature. Proteins were resolved on SDS polyacrylamide gel and transferred to PVDF membrane. Membranes were blocked for 1 hour in 5% non-fat dry milk, 10 mM Tris-HCl pH 7.5, 150 mM NaCl, 0.1% Tween 20 and probed for 2 hours with primary antibodies diluted in blocking buffer. Membranes were than washed 4 times with washing buffer (10 mM Tris-HCl pH 7.5, 150 mM NaCl, 0.1% Tween 20) and incubated for 1 hour with horseradish peroxidase-labeled secondary antibody.

RNase protection assay. RNase protection assays were performed with hApo Multi-Probe Template Sets (BD Biosciences) according to manufacturer's protocol. Briefly, 5 μg of total RNA, collected from each of the exponentially growing cell lines, was hybridized overnight to ³²β-labeled riboprobes, produced on the templates included in the sets. Unhybridized probes were digested by RNaseA. Probes, protected by hybridization to cellular RNA, were resolved on 5% sequencing gel and detected by autoradiography. Expression of the apoptosis-related genes was normalized to that of housekeeping genes

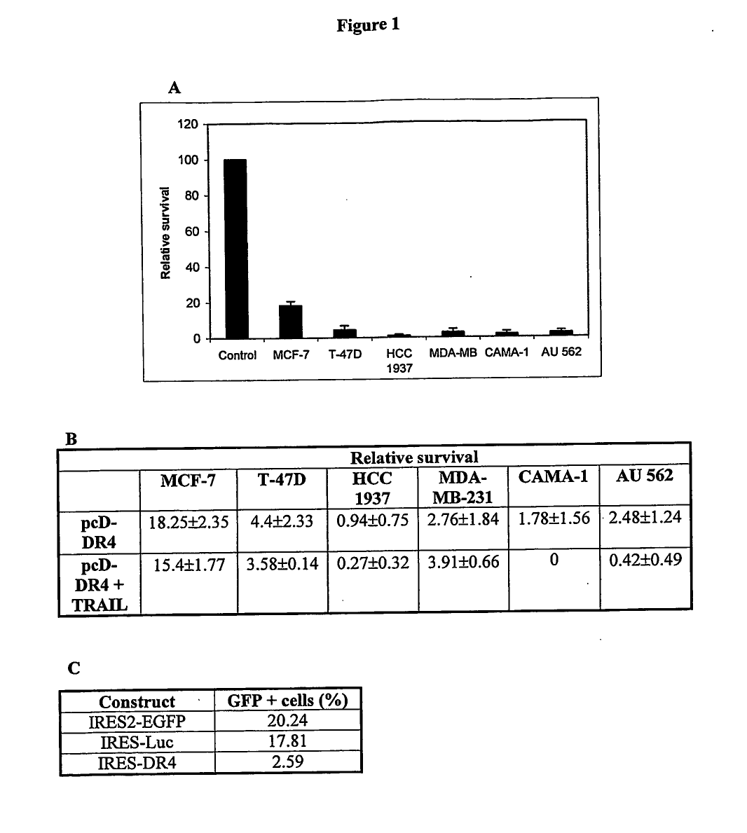

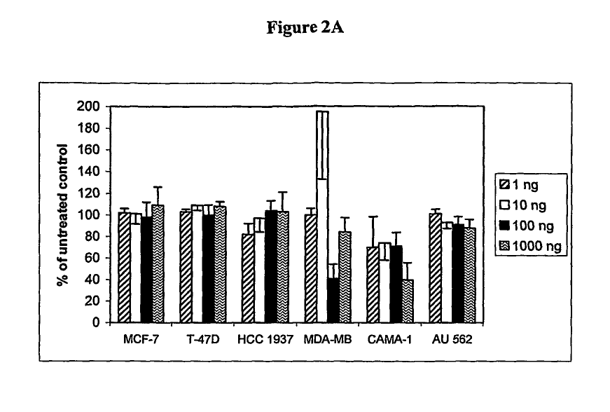

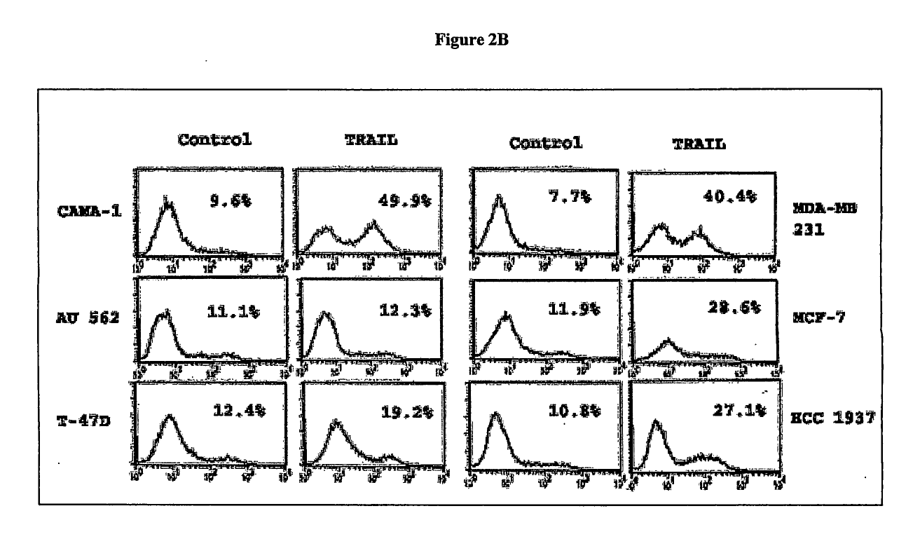

CMV promoter/enhancer-guided DR4 efficiently kills breast cancer cells. To assess the killing efficiency of DR4, two approaches were employed. In the first, six breast cancer cell lines (MCF-7, MDA-MB-231, T-47D, HCC 1937, AU 562 and CAMA-1) were co-transfected with beta-galactosidase reporter gene together with DR4 under the control of a CMV promoter/enhancer (pcD-DR4) or empty vector. Survival was measured by reduction of beta-galactosidase activity in DR4-transfected compared to vector transfected cultures (FIG. 1). Introduction of pcD-DR4 decreased cell viability below 5% of the vector-transfected cells in 5 out of 6 cell lines in the absence of TRAIL (FIG. 1A). In the relatively resistant MCF-7 cell line, cell survival ranged between 16 to 21%. Addition of TRAIL further decreased cell survival to below 1% in AU-562, CAMA-1 and HCC 1937 cells, but had no effect in MCF-7, MDA-MB-231 and T47D cell lines (FIG. 1B); As an alternative method to estimate the effect of DR4 expression on cell survival, DR4 or luciferase (as a control) was cloned into vector pIRES2-eGFP (Clontech) (1-DR4 and I-Luc, respectively). Thus, the reporter and the “killer” gene did not have to be co-transfected; they were present on the same construct and translated from the same mRNA. Cell killing was estimated as the decrease in GFP positive cells in I-DR4-transfected cultures as compared with cultures transfected with an empty vector or I-Luc. In CAMA-1 cells, transiently transfected with unmodified vector or with I-Luc construct, 18-21% of cells expressed GFP 48 hours after transfection (FIG. 1C). In I-DR4 transfected populations, the proportion of GFP-positive cells was decreased to 2.6%. To demonstrate that increased expression of DR4 resulted in killing of TRAIL-resistant cells, sensitivity of the cell lines to soluble TRAIL was tested. Cells were treated with increasing concentrations of recombinant TRAIL or PBS control for 48 hours and cell viability was tested by MTS assay. Only two cell lines, MDA-MB-231 and CAMA-1, showed a significant TRAIL-dependent reduction of viability (FIG. 2A). In MDA-MB-231 cells, TRAIL had a biphasic effect, decreasing cell viability at higher doses while increasing cell proliferation and/or survival at lower concentrations. This pro-survival effect is probably due to the fact that low doses of TRAIL are sufficient to activate NF-κB, but insufficient to induce apoptosis (Degli-Esposti et al. (1997) “The novel receptor TRAIL-R4 induces NF-kappaB and protects against TRAIL-mediated apoptosis, yet retains an incomplete death domain” Immunity 7:813-820; Secchiero et al. (2003) “TRAIL promotes the survival and proliferation of primary human vascular endothelial cells by activating the Akt and ERK pathways” Circulation 107:2250-2256.). Further increase of TRAIL concentrations did not enhance cell death in any of the cell lines tested. To confirm results obtained in the MTS test, cells were incubated with 100 ng of TRAIL for 36 hours, stained for Annexin V and analyzed by flow cytometry (FIG. 2B). The results were in general agreement with the data obtained by MTS test, though MCF-7 and HCC 1937 cells, completely resistant to TRAIL-induced apoptosis by MTS, both showed approximately 17% increase in Annexin V staining. Thus, although only two breast cancer cell lines were sensitive to soluble TRAIL, all of them were efficiently destroyed by DR4.

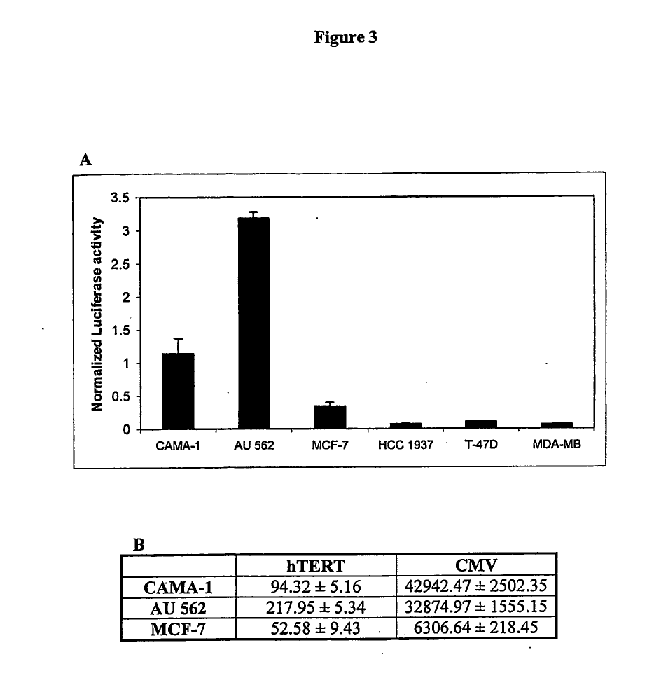

Killing efficiency of hTERT—guided DR4 in breast cancer lines. The efficiency of DR4 in killing cancer cells irrespective of their sensitivity to TRAIL suggested that DR4 might be equally toxic to normal cells. To minimize toxicity to normal cells, expression of this gene was targeted to telomerase positive cancer cells. First hTERT promoter activity was examined in breast cancer cells and compared to the activity of CMV (FIG. 3). In three cell lines, CAMA-1, AU 562 and MCF-7, hTERT promoter activity was relatively high, although significantly (120-450 times) lower than that of the CMV promoter/enhancer (FIGS. 3A, B).

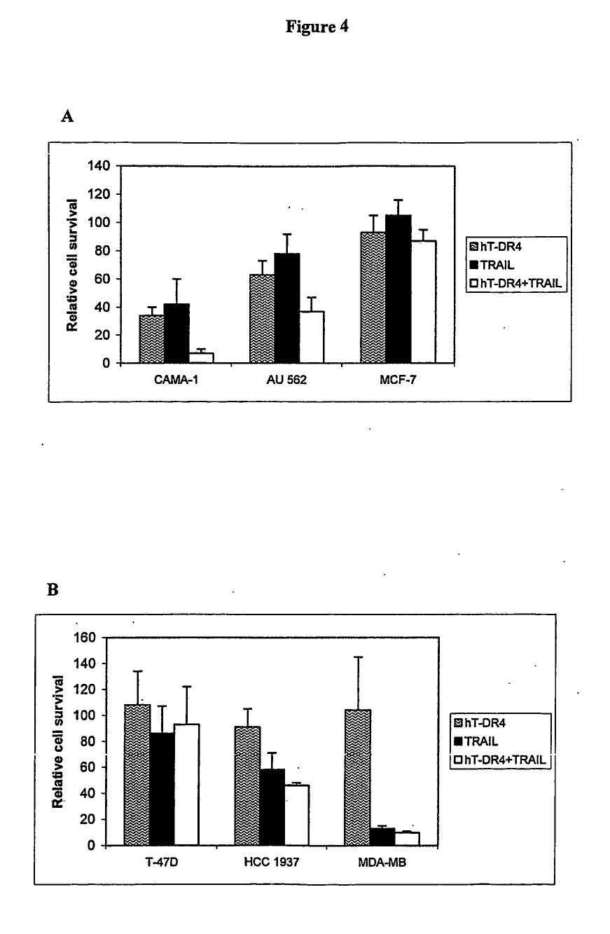

The killing efficiency of hTERT promoter-guided DR4 (hT-DR4) was assessed next. In all cell lines examined, hT-DR4 alone was significantly less effective than pcD-DR4. Among three cell lines with relatively high hTERT promoter activity two (CAMA-1 and AU 562) demonstrated an average of 36% and 63% cell survival respectively (FIG. 4A). Addition of TRAIL at a dose of 100 ng/ml further decreased survival of CAMA-1 cells to an average of 7% and AU 562 cells to an average of 36%. MCF-7 cells, which were relatively resistant to pcD-DR4, did not show a significant degree of cell death. hT-DR4 alone was not toxic to cell lines with low hTERT promoter activity and did not influence cell survival in the presence of TRAIL (FIG. 4B).

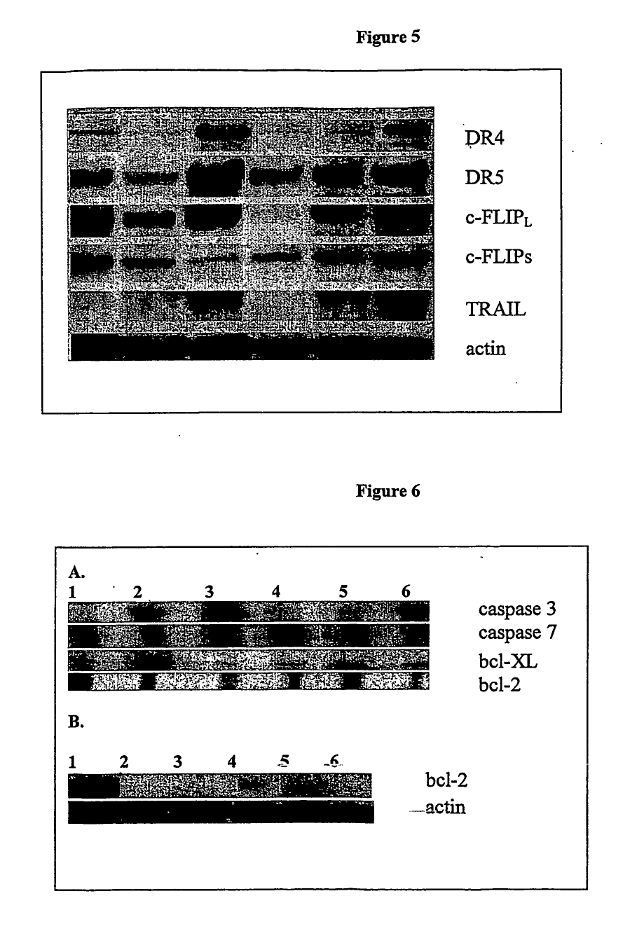

Factors involved in response to hTERT-DR4. These studies indicate that the killing efficiency of hT-DR4 depends on two factors: the activity of hTERT promoter and the resistance to DR4-induced apoptosis. To identify the mechanisms that contribute to resistance to DR4, expression of genes involved in TRAIL-mediated apoptosis in the relatively DR4 resistant MCF-7 cells was compared with the five DR4 sensitive cell lines. The levels of DR4 and DR5 proteins in MCF-7 cells were not significantly lower than in other cell lines under study (FIG. 5 A). MCF-7 cells did not express TRAIL, but this was also true for three out of five sensitive cell lines. MCF-7 cells did express elevated levels of two splice variants of the apoptosis-inhibiting protein FLIP-FLIP_{L} and FLIPs.

MCF-7 cells are deficient in caspase 3 due to a deletion in the gene. This deficiency could account for their inability to undergo apoptosis in response to DR4. To test whether the absence of caspase 3 expression in MCF-7 cells was unique, expression of caspases was evaluated in a panel of breast cancer cell lines by RNase protection assay (FIG. 6). The assay confirmed the absence of caspase 3 expression in MCF-7 cells. However, CAMA-1 cells, which are sensitive to both TRAIL and DR4, did not express detectable levels of caspase 3 mRNA. Another DR4 sensitive cell line, T-47D, expressed a very low level of caspase 3. Of note, these three cell lines expressed high levels of caspase 7, a member of the caspase 3 subfamily, which could substitute for caspase 3 in execution of apoptosis (FIG. 6). There was no difference in expression of caspases 2, 4, 8 and 10A between MCF-7 and the DR4-sensitive cell lines. Caspase 6 expression was lower in MCF-7 cells, but the significance of this finding is uncertain.

Anti-apoptotic members of the bcl-2 family can inhibit death receptor-triggered apoptosis by blocking the apoptotic signal at the level of mitochondria (Fulda et al. (2002) “Inhibition of TRAIL-induced apoptosis by Bcl-2 overexpression” Oncogene 21:2283-2294; Kim et al. (2003) “Functional screening of genes suppressing TRAIL-induced apoptosis: distinct inhibitory activities of Bcl-XL and Bcl-2” Br. J. Cancer 88:910-917), as well as by interfering with processing of caspase 8 (Lamothe and Aggarwal (2002) “Ectopic expression of Bcl-2 and Bcl-xL inhibits apoptosis induced by TNF-related apoptosis-inducing ligand (TRAIL) through suppression of caspases-8, 7, and 3 and BID cleavage in human acute myelogenous leukemia cell line HL-6” J. Interferon Cytokine Res. 22:269-279). Therefore, expression of members of the bcl-2 family were tested by an RNase protection assay. MCF-7 cells expressed bcl-2 mRNA at a much higher level than DR4 sensitive cell lines (FIG. 6A). Bcl-2 overexpression was further confirmed by Western blotting (FIG. 6B). Messenger RNA for other bcl-2 family members, bcl-XL, bclw and mcl-1, was expressed at varying levels in all cell lines and did not correlate with DR4 resistance. MCF-7 cells also did not differ from sensitive cell lines in expression of the members of LAP family of anti-apoptotic proteins, XIAP, survivin, c-IAP 1 and 2 and NAIP.

In summary, this study was an attempt to overcome TRAIL resistance by overexpression of the TRAIL receptor, DR4, based on the concept that overexpression of a functional death receptor will enhance the death stimulus sufficiently to counterbalance the activity of pro-survival pathways and thus will tip the balance towards apoptosis.

Indeed, introduction of DR4 under strong CMV promoter efficiently killed all breast cancer cell lines irrespective of their sensitivity to soluble TRAIL. MCF-7 cells were somewhat more resistant than the other cell lines (average relative cell survival in CMV-DR4 expressing cells 18.3% versus 0.75-4.4% survival obtained with other cells). The ability of DR4 to kill cells in the absence of external TRAIL could be explained by two mechanisms: 1) Cell lines under study themselves produce TRAIL, which than interacts with DR4, and/or 2) High intracellular concentrations of DR4 lead to its ligand-independent oligomerization. As only two cell lines expressed detectable levels of TRAIL on western blot, the first explanation is unlikely to be true. The possibility that high intracellular concentrations of DR4 lead to ligand-independent activation is strengthened by the fact that when expression of DR4 was sufficiently highly driven by the strong CMV promoter, addition of external TRAIL did not significantly increase cell death over DR4 alone. Strong cytotoxicity of DR4 necessitated its transcriptional targeting. For this purpose the promoter of the hTERT gene was used, which has been shown to be selectively active in cancer cells (Gu et al. (2000) “Tumor-specific transgene expression from the human telomerase reverse transcriptase promoter enables targeting of the therapeutic effects of the Bax gene to cancers” Cancer Res. 60:5359-5364; Koga et al. (2000) “A novel telomerase-specific gene therapy: gene transfer of caspase-8 utilizing the human telomerase catalytic subunit gene promoter” Hum. Gene Ther. 11:1397-1406; Komata et al. (2002) “Caspase-8 gene therapy using the human telomerase reverse transcriptase promoter for malignant glioma cells” Hum. Gene Ther. 13:1015-1025; Lin et al. (2002) “Long-term tumor-free survival from treatment with the GFP-TRAIL fusion gene expressed from the hTERT promoter in breast cancer cells” Oncogene 21:8020-8028). Among breast cancer cell lines tested, three (CAMA-1, AU 562 and MCF-7) had relatively high hTERT promoter activity, and these were at least two orders of magnitude lower than that of the CMV promoter. In two of these cell lines, CAMA-1 and AU 562, hTERT-DR4 induced cell death. In accord with promoter activity studies, the degree of cell killing by hTERT-DR4 in these cell lines was considerably lower than that induced by CMV-DR4. However, when cells were treated with a combination of hTERT-DR4 and TRAIL, significant cell killing was achieved. In both cases, the combined effect of hTERT-DR4 and TRAIL was more than additive. Thus, combination of two non-toxic modalities, hTERT-DR4 and TRAIL, efficiently eliminates breast cancer cells with high hTERT promoter activity. No toxicity was observed when cells with low hTERT promoter activity (MDA-MB-23 1, HCC 1937 and T-47D) were treated with hTERT-DR4, with or without TRAIL.

MCF-7 cells were not affected by either hTERT-DR4 alone or by the combination of hTERT-DR4 with TRAIL, despite a comparatively high hTERT promoter activity. This is not surprising, as these cells were also relatively resistant to CMV-DR4. To begin identifying the mechanisms of their resistance, a comparison was made of expression of the members of several families of apoptotic regulators between MCF-7 cells and cell lines sensitive to DR4 overexpression. Among the panel of apoptotic regulators tested, two genes, bcl-2 and FLIP, were expressed at a higher level in MCF-7 cells as compared to the DR4-sensitive cell lines. both bcl-2 and FLIP have been shown to interfere with TRAIL-induced apoptosis (Fulda et al. (2002) “Inhibition of TRAIL-induced apoptosis by Bcl-2 overexpression” Oncogene 21:2283-2294; Hietakangas et al. (2003) “Erythroid differentiation sensitizes K562 leukemia cells to TRAIL-induced apoptosis by downregulation of c-FLIP” Mol. Cell. Biol. 23:1278-1291; Kim et al. (2002) “An inducible pathway for degradation of FLIP protein sensitizes tumor cells to TRAIL-induced apoptosis” J. Biol. Chem. 277:22320-22329; Lamothe and Aggarwal (2002) “Ectopic expression of Bcl-2 and Bcl-xL inhibits apoptosis induced by TNF-related apoptosis-inducing ligand (TRAIL) through suppression of caspases-8, 7, and 3 and BID cleavage in human acute myelogenous leukemia cell line HL-60” J. Interferon Cytokine Res. 22:269-279; xiao et al. (2002) “Tumor necrosis factor-related apoptosis-inducing ligand-induced death-inducing signaling complex and its modulation by c-FLIP and PED/PEA-15 in glioma cells” J. Biol. Chem. 277:25020-25025) and are good candidates for DR4 resistance genes. Expression of BclXL, another bcl-2 family member implicated in TRAIL resistance (Kim et al. (2003) “Functional screening of genes suppressing TRAIL-induced apoptosis: distinct inhibitory activities of Bcl-XL and Bcl-2” Br. J. Cancer 88:910-917; Lamothe and Aggarwal (2002) “Ectopic expression of Bcl-2 and Bcl-xL inhibits apoptosis induced by TNF-related apoptosis-inducing ligand (TRAIL) through suppression of caspases-8, 7, and 3 and BID cleavage in human acute myelogenous leukemia cell line HL-60” J. Interferon Cytokine Res. 22:269-279.), did not show correlation with resistance to DR4. There was also no correlation between DR4 resistance and expression of caspases, IAP and other bcl-2 family members.

### Example 2

### tBid Studies

In this study, tumoricidal activity of tBid and the ability of survivin, hTERT and Muc1 promoters to direct tBid expression in breast cancer cells were evaluated.

Cell lines and reagents. Human breast cancer-cell lines MCF-7, CAMA-1, HCC 1937, MDA-MB-231, T-47D and AU 562 were obtained from the American Type Culture Collection (ATCC). Cells were maintained in RPMI 1640 (T-47D, AU-562 and HCC 1937), α-MEM (MCF-7 and CAMA-1) or Leibovitz's-L15 (MDA-MB-231) medium with 10% FBS, penicillin (100 U/ml) and streptomycin (100 μg/ml) at 37° C. Except for MDA-MB-231, which did not require CO₂ supplementation, all cell lines were grown in humidified atmosphere with 5% CO₂.

Reagents used in the study were obtained from the following companies: FuGENE 6 Transfection Reagent—Roche Applied Science (Indianapolis, Ind.); Dual-Luciferase Reporter Assay System—Promega (Madison, Wis.); Luminescent beta-gal detection kit—BD Biosciences Clontech (Palo Alto, Calif.); Anti-survivin polyclonal antibody—Novus Biologicals (Littleton, Colo.); Mouse anti-Muc1 antibody—Research Diagnostics, Inc. (Flanders, N.J.); Donkey Anti-Rabbit and anti-mouse IgG peroxidase-labeled antibody and ECL™ Western Blotting Detection Reagents—Amersham Biosciences (Little Chalfont, Buckinghamshire, UK); Annexin V-PE apoptosis detection kit—BD Biosciences (Palo Alto, Calif.).

Plasmids and cloning. pRL-TK and pSV40-beta-galactosidase were purchased from Promega hAPR/Noxa and Bik genes were obtained from InVivogen (San Diego, Calif.). DR4, Bid and Bax cDNA were obtained from the ATCC Mammalian Gene Collection and subcloned into pcDNA3.1+ and IRES2-EGFP vectors to produce CMV-DR4, -Bid, -Smac and -Bax and IRES-DR4, -Bid, -Smac and -Bax.

tBid (amino acids 60-195, bases 322-732) was amplified by PCR using the following primers:

| | |
|---|---|
| forward | 5′gccgccatg GATGGCAACCGCAGCAG; |
| reverse | 5′GGTCTTTACCCTACCTGACT. |

The forward primer included Kozac sequence/ATG for optimal translation (indicated in lower case). The resultant PCR product was cloned into T-Vector™ (Promega), verified by sequencing and subsequently subcloned into pcDNA3.1+ and IRES2-EGFP vectors to produce CMV-tBid and IRES-tBid, respectively.

pp 76, containing the hTERT promoter from position −1375 to position +77 (relative to the transcription start site) in the pGL3 Basic vector (containing the firefly luciferase coding sequence) was kindly provided by Dr. Kyo. It was cut on Mlu I/Pst I, blunt-ended and re-closed to delete the Xba I site to produce hTERT-Luc. Luciferase activity of the resultant construct, tested in a panel of breast cancer cell lines, did not differ from that of pp 76. The Survivin promoter (nucleotides 1821-2800, accession number U75285) was amplified by PCR from HELA genomic DNA using the following primers:

| | |
|---|---|
| forward: | 5′-CTGGCCATAGAACCAGAGAAGTGA-3′ |
| reverse: | 5′-CCACCTCTGCCAACGGGTCCCGCG-3′ |

The PCR product was cloned into T-Vector™ (Promega) and subsequently subcloned into the pGL3-Basic vector to produce Survivin-Luc. Fidelity of PCR cloning was verified by sequencing.

The Muc-1 promoter was obtained from InVivogen and subcloned as an NcoI-SpeI fragment into pGL3-Basic to produce Muc-Luc.

hTERT-Luc, Survivin-Luc and Muc-Luc were then restricted on Xba I/Hind III to delete the luciferase gene and tBid, cut out of pcDNA3.1 on Xba I/Hind III, was introduced in its place to produce hT-tBid, Sur-tBid and Muc-tBid, respectively.

To produce CMV promoter-guided luciferase (CMV-Luc) and IRES-Luc, in which luciferase served as a surrogate “killer,” the luciferase gene was cut out of pGL3-Basic and subcloned into pcDNA3.1 and IRES2-EGFP vectors (CMV-Luc and IRES-Luc, respectively).

Transfections and Reporter assays. Transient transfections were performed with FuGENE 6 Transfection Reagent per manufacturer's protocol. Briefly, 5×10⁴ cells per well were plated in 24 well plates and incubated overnight.

For promoter activity studies, promoter-firefly luciferase constructs (100 ng) were co-transfected with pRL-TK (Promega) expressing Renilla luciferase as an internal control, at a ratio of 1:1 into duplicate wells. All assays were performed in duplicate and repeated at least twice. Cells were harvested 36 hours later and a dual luciferase assay was performed using Promega Dual-Luciferase Reporter Assay System per manufacturer's protocol. Results are presented as a ratio of Firefly/Renilla luciferase activity (FL/RL×100%).

For cell survival experiments, two approaches were utilized. In the first one, 5×10⁴ cells in duplicate wells were transfected with 100 ng of a tBid-containing construct together with 100 ng of pSV40-beta-galactosidase reporter gene, which expresses beta-galactosidase under control of the SV40 early promoter. A corresponding construct containing luciferase gene in place of tBid served as a control. For Bik and APR/Noxa genes, which were in the pORF vector backbone, empty pORF vector was used as control. When combinations of the tBid-containing constructs under control of different promoters were examined, 100 ng of each promoter-tBid construct was transfected per well. A combination of the corresponding promoter-luciferase constructs served as control. Beta-galactosidase activity was measured with a luminescent beta-gal detection kit 36 hours later. Relative cell survival was estimated as a percent of beta-galactosidase reporter activity in tBid versus vector-transfected cultures.

In the second approach, cells were transfected with IRES2-EGFP vector, IRES-Luc or IRES-tBid constructs. Thirty-six hours later, expression of GFP was determined by FACS analysis. In other studies, cells were transfected with IRES-Bax, IRES-DR4, IRES-tBID or IRES-Smac. IRES-Luc served as a surrogate killer gene control. After 36 hours, cells were harvested and incubated with phycoerythrin-labeled Annixin-V per manufacturer's protocol. Proportions of GFP- and Annexin V-positive cells were determined by FACS. Relative survival was estimated with the following formula:

$$Relative survival = \frac{\% of GFP ( + ) cells in IRES \text{-} tBID transfected cells \times 100 \%}{\begin{matrix}\% of GFP ( + ) cells in IRES 2 \text{-} EGFP or \\ IRES \text{-} Luc transfected cultures\end{matrix}}$$

Western Blotting. Exponentially growing cells were lysed in lysis buffer (62.5 mM Tris-HCl, pH 6.8, 2% SDS, 1 mM phenylmethylsulfonyl fluoride (PMSF). Whole cell lysates were sonicated to shear released genomic DNA and protein concentrations were determined using a protein assay kit. 25 μg of whole cell protein lysate from each sample was diluted in 25 μl of SDS loading buffer (4% SDS, 2% Glycerol, 0.01% Bromphenol blue, 4% β-mercaptoethanol and 125 mM Tris-HCl, pH 6.8), incubated 5 minutes at 98° C., held for 5 minutes on ice and spun down at maximal speed in a microcentrifuge at room temperature. Proteins were resolved on SDS polyacrylamide gel (6% for Muc1 determination and 10% for survivin determination) and transferred to PVDF membrane. Membranes were blocked for 1 hour in 5% non-fat dry milk in TBST (10 mM Tris-HCl pH 7.5, 150 mM NaCl, 0.1% Tween 20) and probed for 2 hours with primary antibodies diluted in TBST/5% nonfat dry milk per manufacturer's recommendations. Membranes were than washed 4 times with TBST (10 mM Tris-HCl pH 7.5, 150 mM NaCl, 0.1% Tween 20) and incubated for 1 hour with horseradish peroxidase-labeled secondary antibody. Protein expression was then determined by chemiluminescence (ECL, Amersham).

Quantitative PCR. Total RNA was first prepared from cells using the RNAeasy™ kit (Qiagen). CDNA was synthesized using the High Capacity cDNA archive kit (Applied Biosystems). The hTERT and GAPDH (used as an internal reference) Taqman-MGB qPCR sets were obtained as Assays on Deman™ and used and analyzed according to the supplier's protocols (Applied Biosystems).

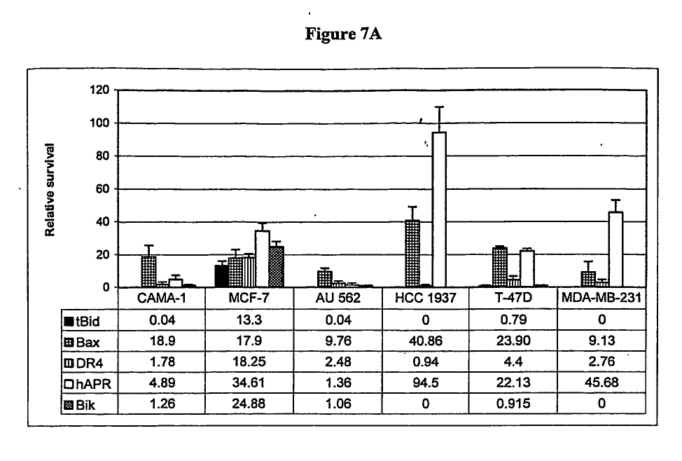

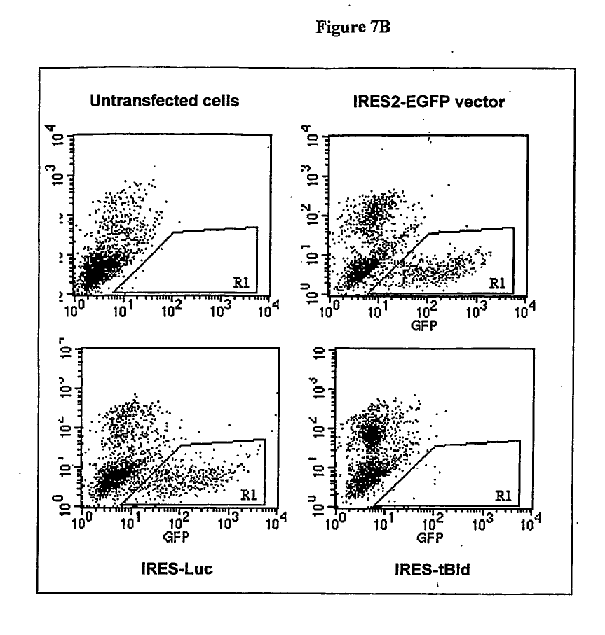

TBid under control of CMV promoter is highly efficient in killing breast cancer cells. The “killing efficiency of tBid, Bax, DR4, HAPR and Bik was compared in co-transfection assays (FIG. 7A). Cells were plated in duplicates and co-transfected with a pro-apoptotic “killer gene” (CMV-tBid) or vector control (CMV-luciferase) together with beta-galactosidase reporter gene at a ratio of 1:1. Beta-galactosidase activity was assayed 48 hours later. Cell survival was estimated as a percentage of beta-galactosidase activity in “killer gene” (i.e., t-Bid)-transfected cells relative to vector (i.e., luciferase)-transfected cultures. Relative cell survival after tBid introduction was below 1% in all cell lines tested except MCF-7 cells. Among the panel tested, tBid was the most efficient killer gene, followed by Bik and DR4. In particular, tumoricidal activity of Bax and Dr4, tested in parallel cultures, was inferior to tBid. Tumoricidal activity of Bik was close to that of tBid, although a direct comparison could not be performed because Bik was under the control of the EF-1/HTLV hybrid promoter, which is reported to be stronger than the CMV promoter. To ensure validity of the results obtained in co-transfection experiments, expression of GFP was assessed in CAMA-1 cells, transiently transfected with plasmids that express pro-apoptotic genes (Bax, DR4, tBid and Smac) and GFP as bicistronic messages (i.e., tBid-IRES-EGFP constructs). These plasmids (e.g., the IRES2-EGFP vector) contain an internal ribosome entry site (IRES) downstream from the cloned gene of interest, followed by the green fluorescence protein (GFP) coding sequence. In transfected cells, the gene of interest and the GFP sequence are transcribed together as a single bicistronic mRNA and are translated in parallel. This approach eliminates the need for co-transfection, as both the “killer gene” (tBid) and the reporter gene (GFP) are expressed from the same plasmid and message. Plasmid IRES-Luc, which served as a control, contains the luciferase coding sequence in place of the “killer gene.” At 36 hours post-transfection, cells were analyzed for Annexin V binding (as a marker of apoptosis) and expression of GFP. The proportion of GFP expressing cells decreased in cultures transfected with pro-apoptotic “killer gene”-GFP plasmids, as compared to the luciferase-GFP plasmid. In agreement with the co-transfection experiments, tBid transfected cultures demonstrated the strongest decrease in relative cell survival, as estimated by the percent of GFP-positive cells in the IRES-tBid-transfected cultures relative to cultures transfected with IRES-Luc. There was an increase in the proportion of Annexin V positive apoptotic cells in the “killer gene” transfected cultures. The magnitude of this increase correlated with the decrease in GFP positive cells. The results obtained confirmed that tBid efficiently destroyed breast cancer cells (FIG. 7B). Although detectable, very few cells were positive for both GFP and Annexin V. This most likely indicates that apoptosis is rapidly induced by the expression of these pro-apoptotic factors, with kinetics that do not allow sufficient time for transcription, translation and maturation of the GFP.

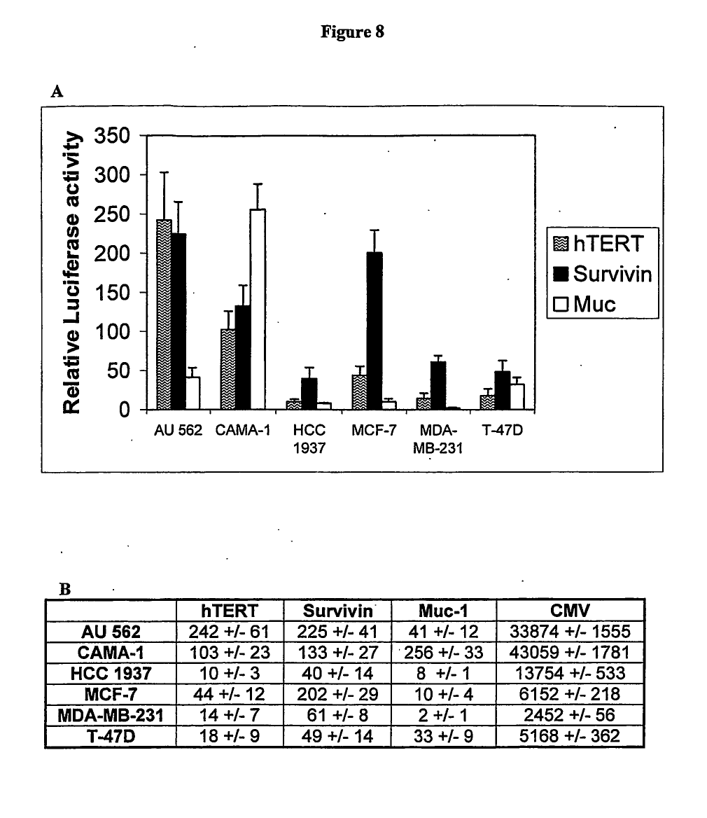

Activity of hTERT, Survivin and Muc1 promoters in breast cancer cells. To target expression of tBid to cancer cells, activity of hTERT, Survivin and Muc-1 promoters were evaluated in breast cancer cells. Promoter activity varied significantly between cell lines (FIG. 8). In all cells tested, activity of tumor-specific promoters was at least two orders of magnitude lower than that of CMV promoter (FIG. 8B). To determine whether promoter activity correlates with expression of the corresponding gene, expression of hTERT, Survivin and Much 1 were evaluated.

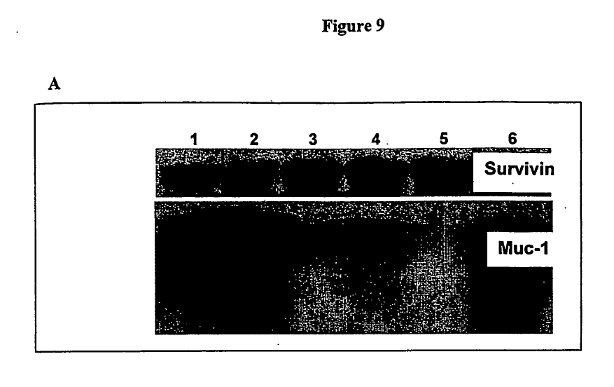

Expression of hTERT mRNA as determined by quantitative PCR was identified in all cell lines tested. There was no strict correlation between the copy number of hTERT mRNA and hTERT promoter activity. For example, HCC1937 and MDA-MB-231 cells had comparable hTERT promoter activity, but expression of hTERT mRNA was five-fold higher in MDA-MB-231 cells. The activity of the hTERT promoter in AU 562 cells was 10× that in mCF-7 cells while the latter cell line had approximately 20% higher hTERT mRNA levels. The most pronounced discrepancy was identified in CAMA-1 cells, which had a very low level of hTERT mRNA while exhibiting high hTERT promoter activity. In this case, the discrepancy could be partially explained by high non-specific background luciferase activity in this cell line (measured by luciferase activity in cultures transfected with a promoter-less pGL3Basic vector), which was on average 10-fold higher than in other cell lines. Muc-1 protein, determined by Western blot analysis, was detected in all cell lines tested, but the level of expression varied from very high in CAMA-1, T-47D and AU 562 cells to barely detectible in MDA-MB-231 cells (FIG. 9). Muc-1 protein levels correlated well with Muc-1 promoter activity, which was high in CAMA-1, T-47D and AU 562 cells and very low in MDA-MB-231 cells.

Survivin protein was expressed in all cell lines tested at comparable levels (FIG. 9A). The same was true for survivin mRNA, as determined by RNase protection assay. Despite the relative uniformity of levels of surviving protein and mRNA, the activity of the Survivin promoter varied up to five-fold between the cell lines.

### Efficiency of tBid Under Control of Tumor-Specific Promoters in Killing Breast Cancer Cells.

To determine whether activity of tumor-specific promoters is sufficient to achieve cell killing with tBid, luciferase was replaced in the hTERT, Muc1 and Survivin constructs with the tBid gene.

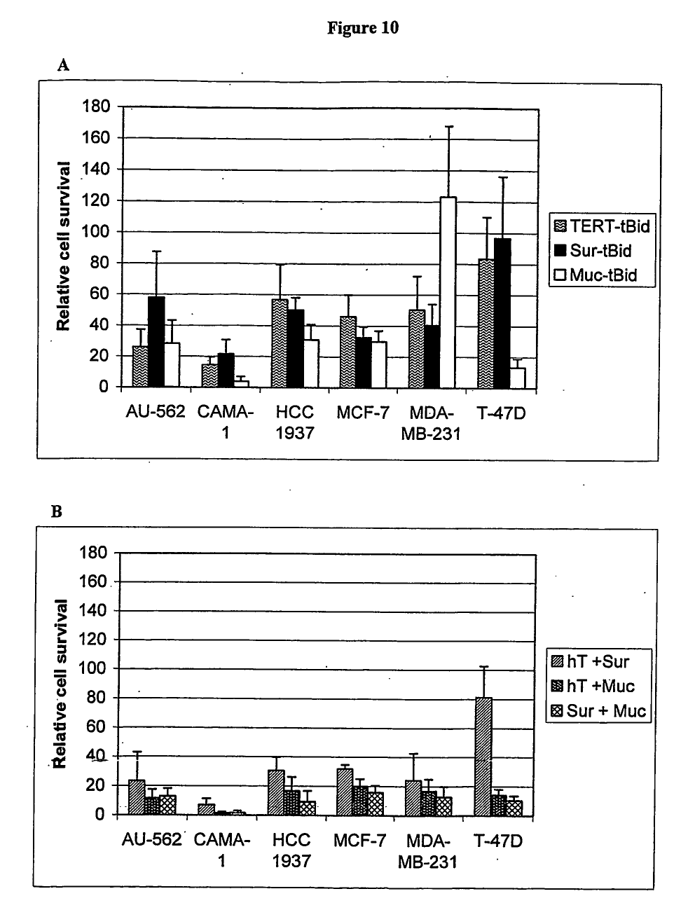

As shown in FIG. 10A, hTERT-tBid efficiently killed CAMA-1 and AU 562 cells, which demonstrated high hTERT promoter activity, was moderately efficient in HCC1937, MDA-MB-231 and MCF-7 cells, and ineffective in T-47D cells, in which hTERT promoter activity was relatively low. The activity of the hTERT promoter was 13-fold lower in T47D cells than in Au 562 cells. The correlation between promoter activity and killing efficiency was not absolute. For example, activity of the hTERT promoter in T-47D cells was higher than in MDA-MB-231 and HCC1937 cells although it did not differ significantly, but the relative cell survival in the former was over 80%, while in the latter two, it was on average below 60%. The same observations were true for the Survivin promoter, which directed significant killing of CAMA-1, MCF-7 and MDA-MB-231 cells, but was inefficient in the T-47D cell line. In AU 562 cells, Sur-tBid was twice less effective than TERT-tBid, despite approximately equal activity of Survivin and hTERT promoters in this cell line.

The Muc-1 promoter was on average more efficient in directing cell killing than hTERT and survivin promoters, despite significantly lower promoter activity. One of the possible explanations for these results is the fact that expression of both hTERT and survivin is cell cycle modulated (Lebeau et al. (2002) “Down-regulation of telomerase activity after progesterone treatment of human breast cancer cells: essential role of the cell cycle status” Anticancer Res. 22:2161-2166; Li et al. (1999) “Pleiotropic cell-division defects and apoptosis induced by interference with survivin function” Nat. Cell Biol. 1:461-466). Therefore, while promoter activity might be very high in cells passing through the “permissive” phase of the cycle, it will be low in the “non-permissive” phases and in non-cycling cells. Muc1-tBid, on the other hand, will kill cells independently of their position in the cell cycle. Of note, cells with low promoter activity (Muc-1 in MDA-MB-231 cells, hTERT and survivin in T-47D cells) were not affected by introduction of tBid under control of this promoter.

To test the combinatorial approach to transcriptional targeting, the effect of simultaneous introduction of tBid under control of different promoters on breast cancer cells was tested (FIG. 10B). Combinations of different promoter-tBid constructs decreased cancer cell survival in an additive manner. Combining two promoter-tBid constructs, which did not kill breast cancer cells when tested individually, did not affect cell survival (see combination of hTERT-tBid with Survivin-tBid in T-47D cells).

In summary, a new gene therapy approach to breast cancer was investigated, with pro-apoptotic gene, tBid, targeted to tumor cells with tumor-specific promoters of hTERT, Survivin and Muc-1 genes.

Comparison of the efficiency of several CMV promoter-guided pro-apoptotic genes in breast cancer cells demonstrated that tBid, an active fragment of the pro-apoptotic gene Bid, induced the highest degree of cell destruction.

To target expression of tBid to breast cancer cells, it was placed under the control of promoters preferentially active in tumor cells: those of Survivin, hTERT and Muc1. Promoter studies confirmed their specificity in tumor cells and the elevated expression of their corresponding proteins in a high proportion of breast tumors. The magnitude of activity varied among cell lines.

tBid under the control of these tumor-specific promoters significantly decreased survival of transfected cells. For each cell line, the degree of cell death correlated with the activity of the corresponding promoter. Our studies also indicate that combinations of different promoter-tBid constructs decrease cancer cell survival in an additive manner.

### Example 3

### Targeting Cells with Loss of Functional p53

Assembling the construct for targeting cells with loss of functional p53. A construct was prepared having the following order of elements:

Lox-poly-A-“killer gene”-promoter Lox-mCre gene-p53-RE

As described herein, targeting of “killer genes” to cancer cells with loss of functional p53 involves assembling a construct where modified Cre gene is placed under the control of p53 response element and a “killer” or surrogate “killer” gene is positioned between two Lox sites.

Cloning and selection of an efficient p53 response element. The first step was to obtain a p53 response element with low background activity and high inducibility by wild type p53 to selectively express Cre recombinase in p53 positive cells. To achieve this goal, pGL3 basic vector (Promega) was modified by cloning a synthetic TATA box upstream of the luciferase gene (pGL3-Bm). Oligonucleotides containing 2 copies of consensus p53 binding site (53C series) or 1 copy of p53 binding site from mdm2 gene (53M series) flanked by Sac I restriction sites were synthesized and cloned at different molar ratios into the Sac I site of modified pGL3-Bm upstream of the TATA box. Clones containing inserts were isolated and promoter strength was tested in a transient transfection assay in the presence of wild type p53. p53C#18, which demonstrated the highest induction of reporter activity by wild type p53, was selected for further studies.

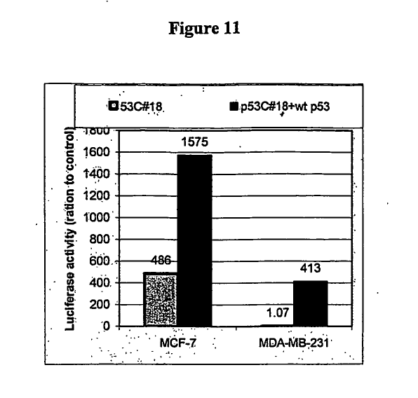

To assess specificity, 53C#18 was tested in MCF-7 cells expressing wild type p53 and in MDA-MB-231 cell lines with p53 mutations. Briefly, equal amounts of p53RE#18 and pRL-TK were administered per well. A Dual Luciferase Reporter assay (Promega) was performed 36-48 hours later in the same sample and transfection efficiencies were normalized. All experiments were performed in duplicate and expressed as a ratio to promoterless control. (FIG. 11). In MDA-MB-231 cells, baseline activity of 53C#18 did not exceed that of promotorless control, and was induced 413 times by co-transfection with wild type p53. MCF-7 cells demonstrated high baseline activity of p53 response element, which was further increased by exogenous p53.

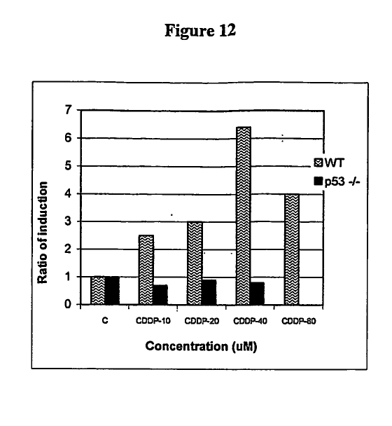

To determine whether treatment with cisplatin (which increases steady-state level of p53 protein) can enhance activity of the p53-RE in normal cells, wild type and p53−/− mouse embryonic fibroblasts were treated with increasing concentrations of cisplatin at the time of transfection (FIG. 12). Cisplatin enhanced activity of p53C#18 in cells with wild type p53 in a dose-dependent manner.

Cre recombinase under the control of p53-RE selectively decreases activity of Luciferase gene flanked by Lox sites in the presence of wild type p53. A gene encoding modified Cre recombinase, provided by Dr. Jeffrey Green, was excised with Xba I/Xho I and cloned into Xba I/Xho I sites downstream of the pre-selected 53C#18/TATA element, in place of luciferase (p53mCre).

Oligonucleotides containing Lox sequences and SV-40 poly-A from IRES2-EGFP vector were cloned into pBSK+ vector (Stratagene) to make pLox plasmid. The luciferase gene, representing a surrogate killer gene, linked to tetracycline response element (Clontech), was introduced between Lox sites (pLox/LucT). The rationale for original selection of TRE as a promoter element was to be able to suppress expression of the killer gene until it is destroyed by Cre. However, TRE demonstrated relatively high background expression in the absence of tetracycline transactivator, and other promoter elements are currently being evaluated.

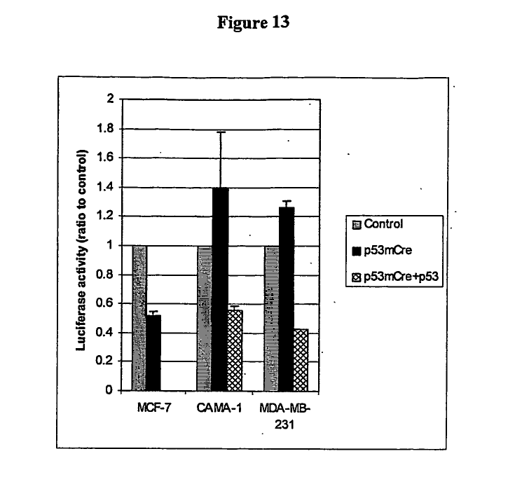

To demonstrate that Cre gene under control of p53-RE was capable of selectively excising a surrogate killer gene in the presence of wild type p53, the pLox/LucT construct was co-transfected with tetracycline transactivator into MCF-7, CAMA-1 and MDA-MB-231 cells, with or without p53mCre, in the presence or absence of exogenous p53 (FIG. 13).

In MCF-7 cells, which express wild type p53, p53mCre caused a decrease of luciferase activity. In p53 negative CAMA-1 and MDA-MB-231 cells, a decrease in luciferase activity was demonstrated only in the presence of exogenous p53. These results confirm selective elimination of the surrogate “killer gene” in cells expressing wild type p53 and its persistence in cells with loss of p53 transcriptional function.

These studies show the placement of a modified Cre gene under the control of a pre-selected p53 response element, (p53mCre) and demonstrate that activity of the luciferase gene, positioned between LoxP sites, is decreased when co-transfected with p53mCre in cells with wild type, but not mutated p53 gene. A targeting construct with luciferase as surrogate “killer gene” (pLox\Lucp53mCre) is also being prepared.

### Example 4

### Effect of tBid in Pancreatic Cancer Cells

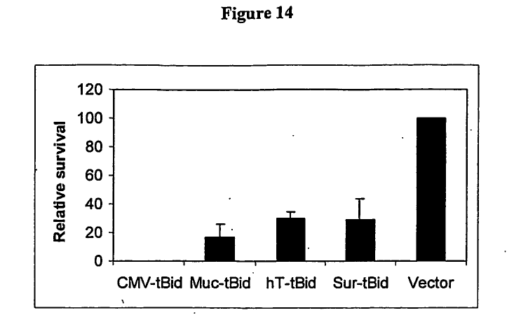

Targeting tBid expression to tumor cells with hTERT, Survivin and Muc-1 promoters. The results obtained in breast cancer cells prompted an evaluation of the efficiency of tBid in pancreatic cancer cells. In co-transfection experiments, MIA-PaCa-2 cells were completely destroyed after introduction of CMV-guided tBid (FIG. 14).

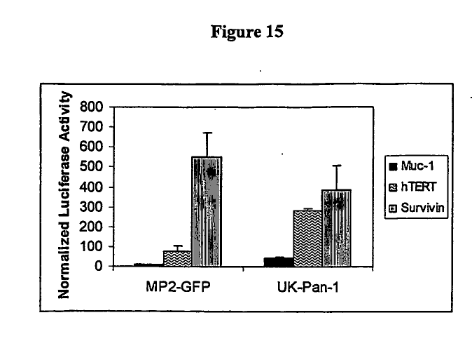

The activity of hTERT, Survivin and Muc-1 promoters was also evaluated in MIA-PaCa-2 and UK-Pan-1 cells. Of the three promoters tested, Survivin had the highest activity, while activity of Muc-1 promoter was relatively low (FIG. 15).

### Example 5

### AAVs as Delivery Vehicles

Recombinant AAVs have several advantages over the currently used systems as vectors suitable for further clinical use: they are non-pathogenic to humans, naturally replication-deficient, do not induce significant immune response, efficiently infect primary tumor cells and penetrate into the center of solid tumors.

tBid was selected because in preliminary studies it was shown to be the most efficient “killer” among a panel of pro-apoptotic genes. Promoters of Survivin, hTERT and Muc1 were selected for their documented tumor-specificity and high prevalence of overexpression of their corresponding genes in tumors of different origins. Activity of these promoters in pancreatic cells was confirmed in preliminary studies.

Plasmids and cloning strategies. Survivin promoter was amplified by PCR and cloned into pGL3 Basic (Promega). hTERT promoter (−1375)/pGL3 Basic construct was a kind gift of Dr. Kyo. Muc1 promoter was obtained from InVivogen and subcloned into pGL3 Basic. tBid (amino acids 60-195, base pairs +322+732 of the Bid gene) was amplified by PCR from a plasmid containing complete coding sequence of Bid gene (ATCC, IMAGE clone). The forward primer contained Kozak sequence/ATG to optimize translation. The resultant PCR product was cloned into pcDNA3 vector and verified by sequencing. It was subsequently subcloned into PGL3 Basic under control of Survivin, hTERT or Muc1 promoters.

AAV helper-free system. Cloning will be carried out by placing tBid or dsRed into the multiple cloning site of pAAV-MCS to produce positive control plasmids (e.g., plasmids in which tBid and dsRed are under the control of a CMV promoter). The CMV promoter will be deleted and the plasmids will either be re-closed to produce promotorless tBid and dsRed for negative controls, or the CMV promoter will be replaced with Survivin, hTERT or Muc1 promoters.

Packaging and titration of recombinant AAVs. Recombinant AAV expression plasmids produced in the step above will be transfected into an AAV-293 packaging cell line together with the pAAV-RC (carrying AAV capsid and replication proteins) and pHelper (harboring adenoviral E2A, E4 and VA genes) by a calcium phosphate precipitation method. The packaging cell line stably expresses adenoviral E1 gene, which completes requirements for production of viral particles. Approximately three days post-transfection (the precise timing is to be determined) supernatants as well as cell lysates prepared by three freeze-thaw cycles will be collected. Titration of the viral stocks will be performed by real time PCR. For in vitro experiments, rAAVs will be produced in a total volume of 30-50 ml each. In vivo experiments will require large-scale rAAV preparations and concentration by CsCl centrifugation. These large scale preparations will be performed for rAAVs, selected for in vivo experiments due to their killing efficiency.

If tBid interferes with rAAV production, its expression will be silenced in the packaging cells with siRNA. This will be accomplished by stable transfection of the packaging cells with tBid siRNA DNA vector (for example, pGE vector from Stratagene) with subsequent selection of tBid-resistant clones. Alternatively, pGE-si-tBid could be included in a co-transfection together with recombinant pAAV expression plasmids, pHelper and pAAV-RC. Other possibilities include expression of high levels of bcl-2 gene, which could protect packaging cells from tBid-induced apoptosis, and packaging rAAVs in vitro. Determination of transduction efficiency of rAAVs in pancreatic cancer cells. Each cell line will be transduced with rAAV containing fluorescent dsRed under control of a CMV promoter with increasing multiplicity of infection (MOI). The proportion of cells expressing dsRed will be determined by flow cytometry. rAAV containing promotorless dsRed will serve as negative control.

It has been shown that γ- and UV irradiation, as well as genotoxic drugs, enhance transduction efficiency, second strand synthesis and transgene expression several fold both in vitro and in vivo. This approach could be used in these studies and can have several added therapeutic benefits. First, in multiple studies, pro-apoptotic genes were shown to work synergistically with chemotherapy and radiation. Second, infection with unmodified AAV2 virus was shown to sensitize cancer cells to chemotherapeutic agents. Sensitization is thought to be due to the protein in the capsid and therefore should be retained by rAAVs. Thus, genotoxic agents will simultaneously increase transgene expression and cooperate with tBid in inducing apoptosis in cancer cells, and AAV infection will enhance sensitivity of pancreatic cells to chemotherapy. Irinotecan will be used as a chemotherapeutic agent for two reasons: 1) topoisomerase inhibitors were shown to be active in the induction of AAV transduction and transgene expression, and 2) irinotecan demonstrated effectiveness against pancreatic cancer in several clinical trials. A range of irinotecan doses will be tested to identify conditions that result in optimal transgene expression.

If transduction efficiency of adeno-associated viruses in pancreatic cells remains low despite all optimization efforts, lentiviral vectors will be used as DNA delivery vehicles. Lentiviral vectors demonstrated high transduction efficiency in multiple tissues, including endocrine pancreas.

Evaluation of activity of Survivin, Muc-1 and hTERT promoters and expression of their corresponding genes. The main goal of this study is to determine activity of the chosen promoters on a single cell level. In preliminary studies luciferase reporter was used to assess promoter activity on the level of a population. This approach does not allow a determination of whether a promoter is equally active in all cells within the population. High promoter activity in a small fraction of cells and moderate promoter activity in all cells will result in comparable total luciferase activity. This knowledge will help in devising an appropriate approach to transcriptional targeting: if promoter activity is equal in all cells, this promoter could be used individually to drive tBid expression. If, however, each promoter is only active in a fraction of cells, constructs carrying tBid under control of different promoters should be used simultaneously or sequentially to target all the cells in a population.

Pancreatic cells will be infected at optimal MOI with AAVs carrying the dsRed gene under control of Survivin, hTERT or Muc-1 promoters (Survivin-dsRed, hTERT-dsRed and Muc1-dsRed). CMV-dsRed rAAV will be used as a control for infection efficiency. At 72 hours post infection, cells will be evaluated for dsRed expression with flow cytometry. The fraction of the cells positive for promoter activity will be estimated using the formula below:

$$Positive fraction = \frac{\begin{matrix}Fraction of dsRed ( + ) cells in \\ promoter \text{-} dsRed \text{-} infected cultures\end{matrix} \times 100 \%}{\begin{matrix}Fraction of dsRed ( + ) cells in \\ CMV \text{-} dsRed \text{-} infected cultures\end{matrix}}$$

A correlation of promoter activity with the expression of the corresponding gene will confirm specificity of promoter activity. It will also allow for the use of corresponding gene expression as a predictive marker of promoter activity, which could be utilized in the clinic to select promoters to treat an individual patient's-tumor.

Expression of Survivin and Muc-1 genes will be evaluated with Western blotting. 30 μg of whole cell protein lysate will be resolved on polyacrylamide gels (10% for Survivin and 6% for Muc-1, which has a molecular mass of 260 to 450 kD). After protein transfer, filters will be incubated with the corresponding primary and secondary antibodies and subjected to ECL.

Expression of hTERT will be evaluated by TaqMan Real-time PCR.

Characterization of the efficiency of the tBid-carrying rAAVs, individually and in combinations, in induction of apoptosis in a panel of pancreatic cancer cell lines. Cells will be infected at increasing MOIs with Survivin-tBid, hTERT-tBid and Muc1-tBid rAAVs and combinations thereof. Promotorless tBid as well as Survivin-dsRed, hTERT-dsRed and Muc1-dsRed AAVs will be used as negative controls. In addition to serving as negative controls, the dsRed-containing AAVs will also allow a determination of the activity of tumor-specific promoters in parallel cultures. Induction of apoptosis in response to rAAV will be determined by acquisition of Annexin V staining and by caspase activation. If transgene expression is insufficient to cause significant apoptosis, infection with control and tBid-carrying rAAVs will be performed in the presence of irinotecan as described herein.

To test whether combinations of rAAVs, which carry tBid under control of different promoters, are superior to the rAAVs used individually, two approaches will be used. In the first approach, the total MOI for the virus will remain constant. For example, if the total MOI for transduction is 300, this MOI will be used for individual promoter-tBid rAAV. When two promoter-tBid rAAVs are employed in combination, each one will be tested at an MOI of 150. For triple combination, the MOI for each rAAV will be 100. In the second approach, each rAAV in the combination will be tested at a dose that produced the best effect when this rAAV was tested individually.

The most effective rAAV or rAAV combination will than be tested in the in vivo models.

### Example 6

### Mouse Model of Human Pancreatic Cancer

Determination of transduction efficiency of rAAV after intra-tumoral and intraperitoneal administration in primary and metastatic pancreatic tumor models. The GFP expression vector pEGFP-N3 (Clontech, Palo Alto, Calif.) was used. GFP expressing MIA PaCa-2 cells were generated by G418 selection after transfection with pEGFP-N3. Transfection of the pEGFP-N3 vector into MIA PaCa-2 was performed using Fugene. At 48 hours after transfection, the cells were subcultured into selective culture medium containing 500 μg/ml G418. GFP expressing cells were isolated and cloned. Clones were further selected for high level of GFP expression, by cell sorting using flow cytometry. The growth rate of these cells was similar to that of parental cells. To produce tumors, MIA PaCa-2 cells will be harvested from subconfluent cultures by a brief exposure to 0.25% trypsin and 0.02% EDTA. Trypsinization will be stopped with medium containing 10% FBS, and the cells will be washed once in serum-free medium and resuspended in HBSS. Suspensions consisting of single cells with >90% viability will be used for injecting into the pancreas of male athymic nude mice. The mice will be anesthetized with ketamine (mouse cocktail, 0.1-0.2 ml/25 g body weight) and the abdomen will be prepped with betadine solution. A longitudinal left paramedian incision will be made in the abdomen and the spleen will be located and mobilized along with the pancreas. Using a 27 gauge needle, a small wheal will be made into the distal pancreas with normal saline.

An inoculum of 3 million pancreatic cancer cells suspended in DMEM will be placed into the wheal. Light pressure will be held over the inoculum site to prevent spillage into the peritoneal cavity. The spleen and pancreas will be returned to their normal anatomical position. The abdomen will then be closed using a combination of 4-0 Nylon sutures and staples. The mice will be returned to their cages and allowed to recover from the anesthesia. If animals show distress during post-op period, they will receive an analgesic (Nalbuphine 4-8 mg/kg IM). The mice will be observed daily for a period of seven days for post operative complications.

Tumor growth will be monitored by fluorescence imaging using the Illumatool Bright Light System LT-9900 from Lightools Research, Encinitas, Calif. The system includes a lamp, power supply, dual Epi lighting, an EGFP filter set 470 nm excitation filter and 51 5 nm viewing filter and is connected to a MagnaFire SP digital Camera (Optronics, Inc. Goleta, Calif.). The anesthetized animal will be placed under the light source and the fluorescence image will be saved to a computer using MagnaFire software.

Determination of transduction efficiency after intratumoral administration of rAAV Two animals will be included in each group for this set of experiments. 4 weeks after tumor cell inoculation, GFP-expressing pancreatic mass will be visualized as described above. Two animals (Group 1) will receive an intratumoral injection of rAAVs carrying LacZ gene and two animals (Group 2) will receive an intratumoral injection of AAV containing dsRed gene under control of a CMV promoter. Both of the genes encode a marker product that can be detected in the tissue and thus will allow identification of cells that express the transgene.

The total injected volume will be 100 μl and a sterile 29-gauge needle will be used. Viral concentration necessary for optimal response will be approximated from the optimal MOIs obtained in the in vitro studies.

If the in vitro experiments indicate that rAAV transduction and transgene expression are improved by irinotecan, separate groups of two animals each will receive an intratumoral injection of rAAV carrying LacZ gene (Group 3) and an intratumoral injection of rAAV containing dsRed gene (Group 4) simultaneously with intraperitoneal irinotecan administration. Doses of irinotecan for intra-peritoneal administration in mouse models vary between 10 and 800 mg/kg. Testing will be initiated at a dose of 100 mg/kg, as it was shown to prolong survival in a mouse model without drug-related toxicity. The dosing, however, can be adjusted according to the extent of augmentation of transgene expression in the in vitro studies. The amount of viral particles per inoculation will also be approximated from the in vitro studies and adjusted as appropriate.

Three days post-injection, animals in all four groups will be euthanized. Tumor tissue will be sampled and examined for LacZ expression with X-Gal staining and a chemiluminescence β-galactosidase activity assay, and for dsRed expression using fluorescence microscopy. A total of eight animals will be used in this experiments. Additional animals might be required if dose adjustments are necessary.

Determination of Transduction Efficiency after Intraperitoneal Administration of rAAVs in Animals with Intraperitoneal Metastases.

After tumor cell inoculation into the pancreas, animals will be imaged weekly to detect development of peritoneal metastases. When presence of macroscopically evident metastases is confirmed, two animals (Group 1) will receive an intra-peritoneal injection of rAAVs carrying LacZ and two animals (Group 2) will receive an intra-peritoneal injection of rAAV carrying dsRed gene under the control of a CMV promoter. As discussed above, two additional groups of animals (Groups 3 and 4) will receive rAAVs together with an intraperitoneal injection of irinotecan, if it proves to be effective in enhancing transgene expression. The amount of viral particles per inoculation will be approximated from the in vitro studies and adjusted as appropriate. A total of eight animals will be used in the initial experiments. Additional animals might be required if dose adjustments are necessary

Characterization of the Efficiency of Targeted tBid in Suppressing Tumor Growth after Repeated Intratumoral Administration of rAAV.

In this group of experiments the most efficient targeted tBid construct or construct combination will be used, as identified during in vitro testing. The number of animals necessary to perform statistical analysis will depend on the magnitude of response to treatment; therefore a pilot experiment using 5 animals per group will be carried out first. Additional animals will be tested to obtain statistically significant data if necessary. Four weeks after tumor cell inoculation, tumors will be visualized and measurements will be performed as described herein. It has been previously shown that tumor measurements obtained in such manner correlate with standard measurements of tumor volume obtained at post-mortem examination. Intra-tumoral injections of rAAVs will be performed twice a week for three weeks. The treatment and control groups will be as follows:
1. Untreated animals
2. Animals treated with intratumoral administration of AAVs carrying targeted tBid
3. Animals treated with intratumoral administration of AAVs carrying targeted DsRed
4. Animals treated with intraperitoneal administration of irinotecan*
5. Animals treated with intratumoral administration of AAVs carrying targeted tBid and intraperitoneal administration of irinotecan*
6. Animals treated with intratumoral administration of AAVs carrying targeted DsRed and intraperitoneal administration of irinotecan*^{#}

### *These groups will be included in the study if irinotecan proves to enhance efficiency of transgene expression and/or tumor cell destruction in the in vitro studies.

### # This group will be incorporated to control for increased tumor sensitivity to irinotecan due to the presence of AAV, unrelated to the activity of “killer gene.”

Tumors will be visualized and measured prior to each treatment and at three weeks. Animals will than be sacrificed and the resultant tumor volume will be confirmed by standard measurements.

A total of 30 animals will be used in this series. Additional animals might be required later to obtain statistically significant results, as discussed above.

Evaluation of the ability of targeted tBid-rAAVs to suppress growth of established peritoneal metastases after repeated intraperitoneal delivery. For these experiments, five animals will be included in each group. Additional animals will be tested to obtain statistically significant data if necessary. After tumor cell inoculation into the pancreas, animals will be imaged weekly to detect development of peritoneal metastases. When presence of macroscopically evident metastases is confirmed, animals will be randomly divided into the following groups:
1. Untreated animals
2. Animals treated with intraperitoneal administration of AAVs carrying targeted tBid
3. Animals treated with intraperitoneal administration of AAVs carrying targeted DsRed (Control).
4. Animals treated with intraperitoneal administration of irinotecan*
5. Animals treated with intraperitoneal administration of AAVs carrying targeted tBid and intraperitoneal administration of irinotecan*
6. Animals treated with intraperitoneal administration of AAVs carrying targeted DsRed and intraperitoneal administration of irinotecan (Control)*

As discussed above, the last three groups will be included in the study if irinotecan proves to be efficient in enhancing transgene expression. Treatments will be repeated twice a week for three weeks. Prior to each treatment, whole body images will be obtained to measure sizes of representative intraperitoneal metastases. After three weeks, animals will be sacrificed and results of non-invasive imaging will be confirmed by autopsy. In addition, multiple organs will be sampled, as well as tumor nodules, from mice receiving targeted dsRed treatment at autopsy. Samples will be immediately frozen in liquid nitrogen and examined for dsRed fluorescence to confirm tumor-specific expression of the transgene.

A total of 30 animals will be used in this series. Additional animals might be required later to obtained statistically significant results, as discussed above.

Investigation of Whether Repeated Introduction of Targeted tBid-rAAVs Prevents Development of Peritoneal Metastases

These experiments will be carried out in essentially the same manner as the ones described above, except treatment will be initiated prior to appearance of macroscopic metastases.

A total of 30 animals will be used in this series. Additional animals might be required later to obtained statistically significant results, as discussed above.

Although the present process has been described with reference to specific details of certain embodiments thereof, it is not intended that such details should be regarded as limitations upon the scope of the invention except as and to the extent that they are included in the accompanying claims.

Throughout this application, various publications are referenced. The disclosures of these publications in their entireties are hereby incorporated by reference into this application in order to more fully describe the state of the art to which this invention pertains.

1. An isolated nucleic acid comprising:
a) a first nucleotide sequence encoding a pro-apoptotic protein selected from the group consisting of DR4, tBid or a biologically active fragment thereof and any combination thereof, and
b) a second nucleotide sequence encoding a tumor-specific promoter selected from the group consisting of survivin promoter, MUC-1 promoter, htert promoter, CEA promoter, PSA promoter, alpha-fetoprotein promoter or a biologically active fragment thereof and any combination thereof, wherein the second nucleotide sequence is operably linked to and directs the expression of the first nucleotide sequence.

2. The nucleic acid of claim 1, further comprising a third nucleotide sequence encoding a pro-apoptotic mediator selected from the group consisting of Bax, Smac, caspase 3, TRAIL or a biologically active fragment thereof and any combination thereof.

3. A vector comprising the nucleic acid of claim 1.

4. The vector of claim 3, wherein the vector is selected from the group consisting of AAV vector, lentivirus vector, adenovirus vector and a nonviral vector.

5. A composition comprising the nucleic acid of claim 1 in a pharmaceutically acceptable carrier.

6. The composition of claim 5, further comprising a chemotherapeutic agent.

7. A method of treating cancer in a subject, comprising administering an effective amount of the nucleic acid of claim 1 to the subject, thereby treating the cancer in the subject.

8. A method of producing a viral vector comprising the nucleic acid of claim 1, comprising introducing into a cell in which viral vectors are produced:
a) a nucleic acid comprising a nucleotide sequence encoding one or more pro-apoptotic proteins, operably linked to one or more tumor-specific promoters and a viral packaging signal;
b) an siRNA construct and/or an antisense sequence that targets the nucleotide sequence encoding the one or more pro-apoptotic proteins of (a), either prior to, or simultaneously with introducing the nucleic acid of (a) into the cell; and
c) nucleic acid encoding viral structural proteins and/or nonstructural proteins necessary to produce viral structural proteins that package nucleic acid into a viral vector particle, under conditions whereby the nucleic acid of (a) is packaged into a viral vector particle, thereby producing the viral vector comprising the nucleic acid of claim 1.

9. A method of producing a viral vector comprising the nucleic acid of claim 1, comprising introducing into a cell in which viral vectors are produced:
a) a nucleic acid comprising a nucleotide sequence encoding one or more pro-apoptotic proteins, operably linked to one or more tumor-specific promoters and a viral packaging signal;
b) a nucleic acid encoding bcl-2 under the direction of an active promoter, either prior to, or simultaneously with introducing the nucleic acid of (a) into the cell; and
c) nucleic acid encoding viral structural proteins and/or nonstructural proteins necessary to produce viral structural proteins that package nucleic acid into a viral vector particle, under conditions whereby the nucleic acid of (a) is packaged into a viral vector particle, thereby producing the viral vector comprising the nucleic acid of claim 1.

10. A vector comprising the nucleic acid of claim 2.

11. A composition comprising the nucleic acid of claim 2 in a pharmaceutically acceptable carrier.

12. The composition of claim 11, further comprising a chemotherapeutic agent.

13. A method of treating cancer in a subject, comprising administering an effective amount of the nucleic acid of claim 2 to the subject, thereby treating the cancer in the subject.

14. A method of treating cancer in a subject, comprising administering an effective amount of the vector of claim 3 to the subject, thereby treating the cancer in the subject.

15. A method of treating cancer in a subject, comprising administering an effective amount of the vector of claim 10 to the subject, thereby treating the cancer in the subject.

16. A method of producing a viral vector comprising the nucleic acid of claim 2, comprising introducing into a cell in which viral vectors are produced:
a) a nucleic acid comprising a nucleotide sequence encoding one or more pro-apoptotic proteins, operably linked to one or more tumor-specific promoters and a viral packaging signal;
b) an siRNA construct and/or an antisense sequence that targets the nucleotide sequence encoding the one or more pro-apoptotic proteins of (a), either prior to, or simultaneously with introducing the nucleic acid of (a) into the cell; and
c) nucleic acid encoding viral structural proteins and/or nonstructural proteins necessary to produce viral structural proteins that package nucleic acid into a viral vector particle, under conditions whereby the nucleic acid of (a) is packaged into a viral vector particle, thereby producing the viral vector comprising the nucleic acid of claim 2.

17. A method of producing a viral vector comprising the nucleic acid of claim 2, comprising introducing into a cell in which viral vectors are produced:
a) a nucleic acid comprising a nucleotide sequence encoding one or more pro-apoptotic proteins, operably linked to one or more tumor-specific promoters and a viral packaging signal;
b) a nucleic acid encoding bcl-2 under the direction of an active promoter, either prior to, or simultaneously with introducing the nucleic acid of (a) into the cell; and
c) nucleic acid encoding viral structural proteins and/or nonstructural proteins necessary to produce viral structural proteins that package nucleic acid into a viral vector particle, under conditions whereby the nucleic acid of (a) is packaged into a viral vector particle, thereby producing the viral vector comprising the nucleic acid of claim 2.
